(19) United States
(12) Patent Application Publication
(10) Pub. No.: US 2020/0206196 A1
(43) Pub. Date: Jul. 2, 2020

(54) Antiviral Drugs

(71) Applicant: Commonwealth Scientific and Industrial Research Organisation, Acton, ACT (AU)

(72) Inventors: Cameron Stewart, Belmont (AU); Andrew Bean, Ocean Grove (AU)

(21) Appl. No.: 16/622,035

(22) PCT Filed: Aug. 11, 2017
(86) PCT No.: PCT/AU2017/050854

§ 371 Date: Dec. 12, 2019

(30) Foreign Application Priority Data
Jun. 13, 2017 (AU) 2017902236

(51) Int. Cl. A61K 31/4184; A61P 31/14; A61K 9/00

(52) U.S. Cl. CPC A61K 31/4184; A61K 45/06; A61K 9/0053; A61P 31/14

(57) ABSTRACT

The present disclosure relates to methods of treating or preventing a viral infection in a subject. More specifically, the invention relates to methods of treating or preventing a Mononegavirales viral infection in a subject that comprises administering an effective amount of an angiotensin II signalling inhibitor.

### FIELD OF THE INVENTION

The present invention relates to a method of treating or preventing a viral infection in a subject. More specifically, the invention relates to a method of treating or preventing a Mononegavirales viral infection in a subject, the method comprising administering an effective amount of an angiotensin II signalling inhibitor.

### BACKGROUND OF THE INVENTION

A virus is a small infectious agent that replicates only inside the living cells of an organism. While a virus can infect all life forms, examples of common viral pathogens known to infect humans include the common cold, influenza, chickenpox, and cold sores. Diseases with more serious complications, such as the Ebola virus disease, avian influenza, human immunodeficiency virus and acquired immunodeficiency syndrome (HIV/AIDS), and severe acute respiratory syndrome (SARS) are also caused by viruses. Furthermore, viral infections are an established cause of cancer in humans and other species.

The symptoms of a viral infection can vary from mild to severely debilitating. If left untreated, viral infections can cause death. The most effective medical approaches to viral diseases are vaccinations to provide immunity to infection, and antiviral drugs that selectively interfere with viral replication. Most antiviral therapies are used for specific viral infections, while a broad-spectrum antiviral therapy is effective against a range of viruses.

However, designing safe and effective antiviral drugs is difficult as the virus will use a host organism's cells to replicate. This makes it challenging to find targets for the antiviral drug that would interfere with the virus without simultaneously harming the host organism's cells. With the ongoing emergence of new viral variants, there is an ongoing need to develop new, safe, and effective antiviral therapies.

Consequently, therefore, there is a need for a new method of treating or preventing a viral infection in a subject.

### SUMMARY OF THE INVENTION

The present inventors have identified a novel and alternative method of treating or preventing a viral infection in a subject.

Accordingly, in one aspect there is provided a method of treating or preventing a Mononegavirales viral infection in a subject, the method comprising administering an effective amount of an angiotensin II signalling inhibitor.

In some embodiments, the Mononegavirales viral infection is of a family selected from the group consisting of Pneumoviridae, Rhabdoviridae, Paramyxoviridae, and Filoviridae.

In some embodiments, the Mononegavirales viral infection is selected from the group consisting of Respiratory Syncytial Virus (RSV), Measles Virus (MeV), Hendra Virus (HeV), Nipah Virus (NiV), Avian Metapneumovirus (aMPV), Human Metapneumovirus (hMPV), Mumps Virus (MuV), Newcastle Disease Virus (NDV), Sendai Virus (SeV), Human Parainfluenza Virus 1 (HPIV-1), Maize Mosaic Virus (MMV), Rice Yellow Stunt Virus (RYSV), Lettuce Necrotic Yellow Virus (LNYV), Rabies Virus (RABV), Vesicular Stomatitis Indiana Virus (VSIV), Bovine Ephemeral Fever Virus (BEFV), Infectious Hematopoietic Necrosis Virus (IHNV), Marburg Virus (LVMV), and Ebola Virus (EBOV).

In some preferred embodiments, the Mononegavirales viral infection is Respiratory Syncytial Virus (RSV).

In some preferred embodiments, the Mononegavirales viral infection is Measles Virus (MeV).

In some embodiments, the angiotensin II signalling inhibitor has a structure selected from the group consisting of:

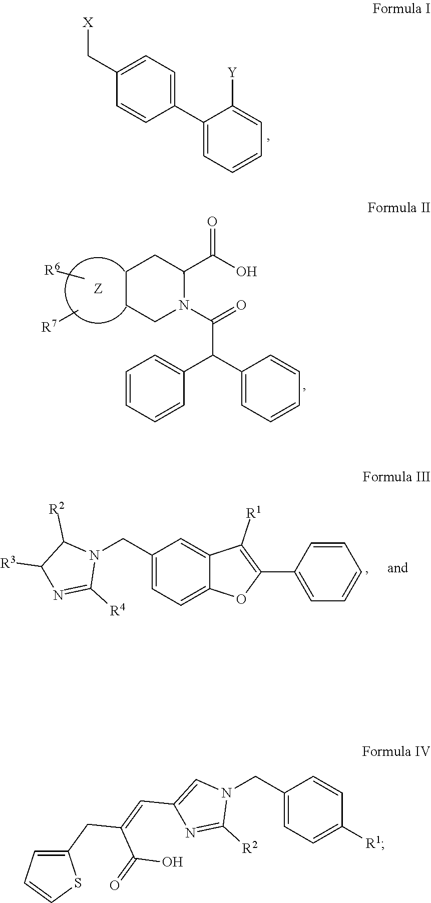

wherein X is selected from the group consisting of:

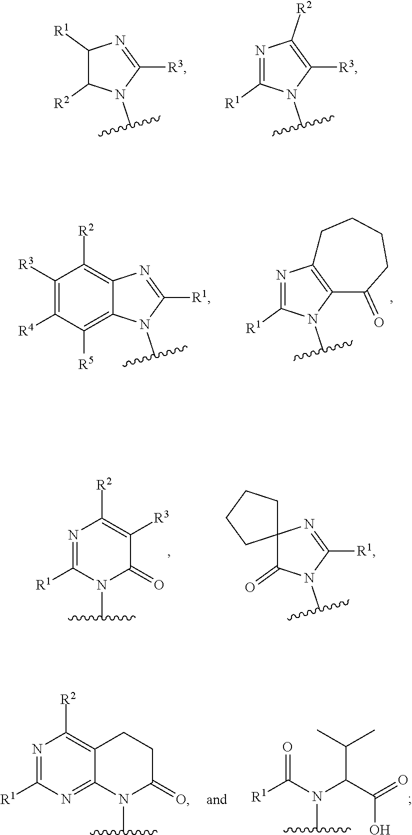

and wherein Y is selected from the group consisting of:

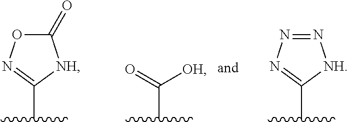

and wherein Z is a 5- or 6-membered monocyclic carbocyclic or monocyclic heterocyclic; and
wherein R¹, R², R³, R⁴, R⁵, R⁶ and R⁷ are each independently selected from the group consisting of hydrogen, halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, monocyclic or polycyclic carbocyclic, and monocyclic or polycyclic heterocyclic;

wherein the C₁₋₁₀alkyl, C₂₋₁₀alkenyl, monocyclic or polycyclic carbocyclic, and monocyclic or polycyclic heterocyclic, are each optionally substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, monocyclic or polycyclic carbocyclic, and monocyclic or polycyclic heterocyclic;

wherein the monocyclic or polycyclic carbocyclic, and monocyclic or polycyclic heterocyclic are each optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, and C₁₋₁₀ alkyl; and

wherein the C₁₋₁₀alkyl and C₂₋₁₀alkenyl are each optionally interrupted with one or more heteroatoms independently selected from O, N and S.

In some embodiments, the angiotensin II signalling inhibitor is an angiotensin II receptor antagonist.

In some embodiments, the angiotensin II signalling inhibitor is an angiotensin II receptor AT₁ subtype antagonist.

In some preferred embodiments, the angiotensin II signalling inhibitor is selected from the group consisting of Telmisartan, Candesartan, Losartan, Valsartan, Eprosartan, Irbesartan, Fimasartan, Saprisartan, Olmesartan, Azilsartan, Pratosartan, Tasosartan, EXP-3174, TCV-116, PD123319, EMA401, and pharmaceutically acceptable salts and solvates thereof.

In some particularly preferred embodiments, the angiotensin II signalling inhibitor is Telmisartan.

In some embodiments, the angiotensin II signalling inhibitor binds to the angiotensin II receptor.

In some embodiments, the angiotensin II signalling inhibitor is an angiotensin-converting enzyme (ACE) inhibitor.

In some embodiments, the angiotensin II signalling inhibitor is a renin inhibitor.

In some embodiments, the angiotensin II signalling inhibitor blocks viral RNA replication.

In some embodiments, the angiotensin II signalling inhibitor is administered in combination with another antiviral compound.

In some embodiments, the subject is not being treated for endothelial dysfunction, hypertension, diabetic nephropathy or congestive heart failure.

In some embodiments, the subject is not being treated for endothelial dysfunction.

In some embodiments, the angiotensin II signalling inhibitor is administered to the subject orally.

In some embodiments, the subject is a bird or mammal.

In some preferred embodiments, the mammal is a human.

In another aspect, there is provided an angiotensin II signalling inhibitor for use in treating or preventing a Mononegavirales viral infection in a subject.

In another aspect, there is provided an antiviral agent selected from an angiotensin II signalling inhibitor for use in treating or preventing a Mononegavirales viral infection.

In another aspect, there is provided the use of an angiotensin II signalling inhibitor for treating or preventing a Mononegavirales viral infection in a subject.

In another aspect, there is provided the use of an angiotensin II signalling inhibitor in the manufacture of a medicament for the treatment or prevention of a Mononegavirales viral infection in a subject.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1—Workflow and controls used for a high throughput chemical screen of inhibitors of HeV infection at BSL-4. (A) A library of 4,148 known drugs spanning libraries from Tocris, Prestwick and Lopac were aliquoted into 384 well tissue culture plates, and incubated with HeLa cells (4,000 cells/well) for 1 h. Cells were then infected with HeV for 24 h at an MOI of 1. Plates were fixed with 4% paraformaldehyde and stained to detect viral antigen and cell viability. (B) Positive control compounds used in this screen, showing their impact on relative cell numbers and HeV infection. (C) Results from the compound screen, with compounds ranked according to relative HeV infection from lowest (decreased virus replication) to highest (increased virus replication).

FIG. 2—Telmisartan inhibits infection by HeV and other negative strand RNA viruses. (A) HeV titres in HeLa cells infected with HeV (MOI 1) for 24 h, 1 h post-treatment with Telmisartan or equivalent DMSO control, *p<0.05. Relative cell numbers (B) and metabolic activity (C) of HeLa cells 26 h post-treatment with Telmisartan or equivalent DMSO control, *p<0.05 compared to siNEG. (D) Virus titres in HeLa cells infected with indicated viruses (MOI 1) for 24 h, 1 h post-treatment with Telmisartan (10 μM), *p<0.05 compared to DMSO control, not significant.

FIG. 3—Telmisartan inhibits the early stages of HeV infection post-entry. (A) Cell-to-cell fusion of HeV-F and HeV-G-expressing (effector) HEK-293T cells to (target) HeLa cells treated with DMSO or Telmisartan (10 μM). Values are normalised to mock, set to 100, *p<0.05 compared to vehicle (DMSO control). (B) qRT-PCR measurements of intracellular viral RNA in HeLa cells and (C) TCID₅₀ measurements of virus titres in cells infected with HeV (MOI 5), *p<0.05 compared to mock. HeV RNA values are normalised to cellular 18S levels and to inoculum levels of HeV, set to 1. (D) Immunofluorescence microscopy showing HeV-P protein staining in HeLa cells transfected with siNEG or siFBL, followed by HeV infection (MOI 0.1, 24 h).

FIG. 4—HeV infection is inhibited by multiple angiotensin II signalling inhibitor. HeV titres in HeLa cells infected with HeV (MOI 1) for 24 h, 1 h post-treatment with Telmisartan, Candesartan or equivalent DMSO control, *p<0.05.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., biochemistry, chemistry, medicinal chemistry, antiviral drug discovery, and the like).

As used herein, the term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−20%, more preferably +/−10%, of the designated value.

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “subject” refers to any organism susceptible to a Mononegavirales viral infection. For example, the subject can be a mammal, avian, arthropod, chordate, amphibian or reptile. Exemplary subjects include but are not limited to human, bird (e.g. chicken, duck), primate, livestock (e.g. sheep, cow, chicken, horse, donkey, pig), companion animals (e.g. dog, cat), laboratory test animals (e.g. mouse, rabbit, rat, guinea pig, hamster), and captive wild animals (e.g. fox, deer). In one example, the subject is a bird. In one example, the subject is a mammal. In one example, the subject is human.

As used herein, the term “treating” includes alleviation of the symptoms associated with a specific disorder or condition and eliminating said symptoms. For example, as used herein, the term “treating a viral infection” refers to alleviating the symptoms associated with a viral infection and eliminating said symptoms.

As used herein, the term “prevention” includes prophylaxis of the specific disorder or condition. For example, as used herein, the term “preventing a viral infection” refers to preventing the onset or duration of the symptoms associated with a viral infection.

As would be understood by the person skilled in the art, an angiotensin II signalling inhibitor would be administered in a therapeutically effective amount. The term “therapeutically effective amount”, as used herein, refers to an angiotensin II signalling inhibitor being administered in an amount sufficient to alleviate or prevent to some extent one or more of the symptoms of the disorder or condition being treated. The result can be the reduction and/or alleviation of the signs, symptoms, or causes of a disease or condition, or any other desired alteraction of a biological system. For example, one result may be the reduction and/or alleviation of the symptom of a fever associated with a viral infection. The term, an “effective amount”, as used herein, refers to an amount of an angiotensin II signalling inhibitor effective to achieve a desired pharmacologic effect or therapeutic improvement without undue adverse side effects. By way of example only, therapeutically effective amounts may be determined by routine experimentation, including but not limited to a dose escalation clinical trial. The term “therapeutically effective amount” includes, for example, a prophylactically effective amount. It is understood that “an effective amount” or “a therapeutically effective amount” can vary from subject to subject, due to variation in metabolism of the compound and any of age, weight, general condition of the subject, the condition being treated, the severity of the condition being treated, and the judgment of the prescribing physician. Thus, it is not always possible to specify an exact “effective amount”. However, an appropriate “effective amount” in any individual case may be determined by one of ordinary skill in the art using routine experimentation. Where more than one therapeutic agent is used in combination, a “therapeutically effective amount” of each therapeutic agent can refer to an amount of the therapeutic agent that would be therapeutically effective when used on its own, or may refer to a reduced amount that is therapeutically effective by virtue of its combination with one or more additional therapeutic agents.

The term “onset” of activity, as used herein, refers to the length of time to alleviate or prevent to some extent one or more of the symptoms of the disorder or condition being treated following the administration of the angiotensin II signalling inhibitor. The term “duration” refers to the length of time that the therapeutic continues to be therapeutically effective, i.e., alleviate or prevent to some extent one or more of the symptoms of the disorder or condition being treated. The person skilled in the art would be aware that onset, peak, and duration of therapy may vary depending on factors such as the patient, the condition of the patient, and the route of administration.

The terms “carbocyclic” and “carbocyclyl” represent a monocyclic or polycyclic ring system wherein the ring atoms are all carbon atoms, e.g., of about 3 to about 20 carbon atoms, and which may be aromatic, non-aromatic, saturated, or unsaturated, and may be substituted and/or contain fused rings. Examples of such groups include aryl groups such as benzene, saturated groups such as cyclopentyl, or fully or partially hydrogenated phenyl, naphthyl and fluorenyl. It will be appreciated that the polycyclic ring system includes bicyclic and tricyclic ring systems.

“Heterocyclyl” or “heterocyclic” whether used alone, or in compound words such as heterocyclyloxy, represents a monocyclic or polycyclic ring system wherein the ring atoms are provided by at least two different elements, typically a combination of carbon and one or more of nitrogen, sulfur and oxygen, although may include other elements for ring atoms such as selenium, boron, phosphorus, bismuth, and silicon, and wherein the ring system is about 3 to about 20 atoms, and which may be aromatic such as a “heteroaryl” group, non-aromatic, saturated, or unsaturated, and may be substituted and/or contain fused rings. For example, the heterocyclyl may be (i) an optionally substituted cycloalkyl or cycloalkenyl group, e.g., of about 3 to about 20 ring members, which may contain one or more heteroatoms such as nitrogen, oxygen, or sulfur (examples include pyrrolidinyl, morpholino, thiomorpholino, or fully or partially hydrogenated thienyl, furyl, pyrrolyl, thiazolyl, oxazolyl, oxazinyl, thiazinyl, pyridyl and azepinyl); (ii) an optionally substituted partially saturated monocyclic or polycyclic ring system in which an aryl (or heteroaryl) ring and a heterocyclic group are fused together to form a cyclic structure (examples include chromanyl, dihydrobenzofuryl and indolinyl); or (iii) an optionally substituted fully or partially saturated polycyclic fused ring system that has one or more bridges (examples include quinuclidinyl and dihydro-1,4-epoxynaphthyl). It will be appreciated that the polycyclic ring system includes bicyclic and tricyclic ring systems.

As will be understood, an “aromatic” group means a cyclic group having 4m+2 π electrons, where m is an integer equal to or greater than 1. As used herein, “aromatic” is used interchangeably with “aryl” to refer to an aromatic group, regardless of the valency of aromatic group.

“Aryl” whether used alone, or in compound words such as arylalkyl, aryloxy or arylthio, represents: (i) an optionally substituted monocyclic or polycyclic aromatic carbocyclic moiety, e.g., of about 6 to about 20 carbon atoms, such as phenyl, naphthyl or fluorenyl; or, (ii) an optionally substituted partially saturated polycyclic carbocyclic aromatic ring system in which an aryl and a cycloalkyl or cycloalkenyl group are fused together to form a cyclic structure such as a tetrahydronaphthyl, indenyl, indanyl or fluorene ring. It will be appreciated that the polycyclic ring system includes bicyclic and tricyclic ring systems.

A “hetaryl”, “heteroaryl” or heteroaromatic group, is an aromatic group or ring containing one or more heteroatoms, such as nitrogen, oxygen, sulfur, selenium, silicon or phosphorus. As used herein, “heteroaromatic” is used interchangeably with “hetaryl” or “heteroaryl”, and a heteroaryl group refers to monovalent aromatic groups, bivalent aromatic groups and higher multivalency aromatic groups containing one or more heteroatoms. For example, “heteroaryl” whether used alone, or in compound words such as heteroaryloxy represents: (i) an optionally substituted monocyclic or polycyclic aromatic organic moiety, e.g., of about 5 to about 20 ring members in which one or more of the ring members is/are element(s) other than carbon, for example nitrogen, oxygen, sulfur or silicon; the heteroatom(s) interrupting a carbocyclic ring structure and having a sufficient number of delocalized π electrons to provide aromatic character, provided that the rings do not contain adjacent oxygen and/or sulfur atoms. Typical 6-membered heteroaryl groups are pyrazinyl, pyridazinyl, pyrazolyl, pyridyl and pyrimidinyl. All regioisomers are contemplated, e.g., 2-pyridyl, 3-pyridyl and 4-pyridyl. Typical 5-membered heteroaryl rings are furyl, imidazolyl, oxazolyl, isoxazolyl, isothiazolyl, oxadiazolyl, pyrrolyl, 1,3,4-thiadiazolyl, thiazolyl, thienyl, triazolyl, and silole. All regioisomers are contemplated, e.g., 2-thienyl and 3-thienyl. Bicyclic groups typically are benzo-fused ring systems derived from the heteroaryl groups named above, e.g., benzofuryl, benzimidazolyl, benzthiazolyl, indolyl, indolizinyl, isoquinolyl, quinazolinyl, quinolyl and benzothienyl; or, (ii) an optionally substituted partially saturated polycyclic heteroaryl ring system in which a heteroaryl and a cycloalkyl or cycloalkenyl group are fused together to form a cyclic structure such as a tetrahydroquinolyl or pyrindinyl ring. It will be appreciated that the polycyclic ring system includes bicyclic and tricyclic ring systems.

The term “optionally substituted” means that a functional group is either substituted or unsubstituted, at any available position. Substitution can be with one or more functional groups selected from, e.g., alkyl, alkenyl, alkynyl, cycloalkyl, cycloalkenyl, aryl, heterocyclyl, heteroaryl, formyl, alkanoyl, cycloalkanoyl, aroyl, heteroaroyl, carboxyl, alkoxycarbonyl, cycloalkyloxycarbonyl, aryloxycarbonyl, heterocyclyloxycarbonyl, heteroaryloxycarbonyl, alkylaminocarbonyl, cycloalkylaminocarbonyl, arylaminocarbonyl, heterocyclylaminocarbonyl, heteroarylaminocarbonyl, cyano, alkoxy, cycloalkoxy, aryloxy, heterocyclyloxy, heteroaryloxy, alkanoate, cycloalkanoate, aryloate, heterocyclyloate, heteroaryloate, alkylcarbonylamino, cycloalkylcarbonylamino, arylcarbonylamino, heterocyclylcarbonylamino, heteroarylcarbonylamino, nitro, alkylthio, cycloalkylthio, arylthio, heterocyclylthio, heteroarylthio, alkylsulfonyl, cycloalkylsulfonyl, arylsulfonyl, heterocyclysulfonyl, heteroarylsulfonyl, hydroxyl, halo, haloalkyl, haloaryl, haloheterocyclyl, haloheteroaryl, haloalkoxy, haloalkylsulfonyl, silylalkyl, alkenylsilylalkyl, and alkynylsilylalkyl. It will be appreciated that other groups not specifically described may also be used.

The term “halo” or “halogen” whether employed alone or in compound words such as haloalkyl, haloalkoxy or haloalkylsulfonyl, represents fluorine, chlorine, bromine or iodine. Further, when used in compound words such as haloalkyl, haloalkoxy or haloalkylsulfonyl, the alkyl may be partially halogenated or fully substituted with halogen atoms which may be independently the same or different. Examples of haloalkyl include, without limitation, —CH₂CH₂F, —CF₂CF₃ and —CH₂CHFCl. Examples of haloalkoxy include, without limitation, —OCHF₂, —OCF₃, —OCH₂CCl₃, —OCH₂CF₃ and —OCH₂CH₂CF₃. Examples of haloalkylsulfonyl include, without limitation, —SO₂CF₃, —SO₂CCl₃, —SO₂CH₂CF₃ and —SO₂CF₂CF₃.

“Alkyl” whether used alone, or in compound words such as alkoxy, alkylthio, alkylamino, dialkylamino or haloalkyl, represents straight or branched chain hydrocarbons ranging in size from one to about 20 carbon atoms, or more. Thus alkyl moieties include, unless explicitly limited to smaller groups, moieties ranging in size, for example, from one to about 6 carbon atoms or greater, such as, methyl, ethyl, n-propyl, iso-propyl and/or butyl, pentyl, hexyl, and higher isomers, including, e.g., those straight or branched chain hydrocarbons ranging in size from about 6 to about 20 carbon atoms, or greater. In one example, the alkyl moiety is of one to 10 carbon atoms.

“Alkenyl” whether used alone, or in compound words such as alkenyloxy or haloalkenyl, represents straight or branched chain hydrocarbons containing at least one carbon-carbon double bond, including, unless explicitly limited to smaller groups, moieties ranging in size from two to about 6 carbon atoms or greater, such as, methylene, ethylene, 1-propenyl, 2-propenyl, and/or butenyl, pentenyl, hexenyl, and higher isomers, including, e.g., those straight or branched chain hydrocarbons ranging in size, for example, from about 6 to about 20 carbon atoms, or greater. In one example, the alkenyl moiety is of two to 10 carbon atoms.

“Alkynyl” whether used alone, or in compound words such as alkynyloxy, represents straight or branched chain hydrocarbons containing at least one carbon-carbon triple bond, including, unless explicitly limited to smaller groups, moieties ranging in size from, e.g., two to about 6 carbon atoms or greater, such as, ethynyl, 1-propynyl, 2-propynyl, and/or butynyl, pentynyl, hexynyl, and higher isomers, including, e.g., those straight or branched chain hydrocarbons ranging in size from, e.g., about 6 to about 20 carbon atoms, or greater. In one example, the alkynyl moiety is of two to 20 carbon atoms.

“Cycloalkyl” represents a mono- or polycarbocyclic ring system of varying sizes, e.g., from about 3 to about 20 carbon atoms, e.g., cyclopropyl, cyclobutyl, cyclopentyl, cyclohexyl or cycloheptyl. The term cycloalkyloxy represents the same groups linked through an oxygen atom such as cyclopentyloxy and cyclohexyloxy. The term cycloalkylthio represents the same groups linked through a sulfur atom such as cyclopentylthio and cyclohexylthio.

“Cycloalkenyl” represents a non-aromatic mono- or polycarbocyclic ring system, e.g., of about 3 to about 20 carbon atoms containing at least one carbon-carbon double bond, e.g., cyclopentenyl, cyclohexenyl or cycloheptenyl. The term “cycloalkenyloxy” represents the same groups linked through an oxygen atom such as cyclopentenyloxy and cyclohexenyloxy. The term “cycloalkenylthio” represents the same groups linked through a sulfur atom such as cyclopentenylthio and cyclohexenylthio.

“Cycloalkynyl” represents a non-aromatic mono- or polycarbocyclic ring system, e.g., of about 3 to about 20 carbon atoms containing at least one carbon-carbon double bond, e.g., cyclopentenyl, cyclohexenyl or cycloheptenyl. The term “cycloalkenyloxy” represents the same groups linked through an oxygen atom such as cyclopentenyloxy and cyclohexenyloxy. The term “cycloalkenylthio” represents the same groups linked through a sulfur atom such as cyclopentenylthio and cyclohexenylthio.

“Alkanoyl” represents a —C(═O)-alkyl group in which the alkyl group is as defined supra. In a particular embodiment, an alkanoyl ranges in size from about C₂-C₂₀. One example is acyl.

“Aroyl” represents a —C(═O)-aryl group in which the aryl group is as defined supra. In a particular embodiment, an aroyl ranges in size from about C₇-C₂₀. Examples include benzoyl and 1-naphthoyl and 2-naphthoyl.

“Heterocycloyl” represents a —C(═O)-heterocyclyl group in which the heterocylic group is as defined supra. In a particular embodiment, an heterocycloyl ranges in size from about C₄-C₂₀.

“Heteroaroyl” represents a —C(═O)-heteroaryl group in which the heteroaryl group is as defined supra. In a particular embodiment, a heteroaroyl ranges in size from about C₆-C₂₀. An example is pyridylcarbonyl.

“Carboxyl” represents a —CO₂H moiety.

“Oxycarbonyl” represents a carboxylic acid ester group —CO₂R which is linked to the rest of the molecule through a carbon atom.

“Alkoxycarbonyl” represents an —CO₂-alkyl group in which the alkyl group is as defined supra. In a particular embodiment, an alkoxycarbonyl ranges in size from about C₂-C₂₀. Examples include methoxycarbonyl and ethoxycarbonyl.

“Aryloxycarbonyl” represents an —CO₂-aryl group in which the aryl group is as defined supra. Examples include phenoxycarbonyl and naphthoxycarbonyl.

“Heterocyclyloxycarbonyl” represents a —CO₂-heterocyclyl group in which the heterocyclic group is as defined supra.

“Heteroaryloxycarbonyl” represents a —CO-heteroaryl group in which the heteroaryl group is as defined supra.

“Aminocarbonyl” represents a carboxylic acid amide group —C(═O)NHR or —C(═O)NR₂ which is linked to the rest of the molecule through a carbon atom.

“Alkylaminocarbonyl” represents a —C(═O)NHR or —C(═O)NR₂ group in which R is an alkyl group as defined supra.

“Arylaminocarbonyl” represents a —C(═O)NHR or —C(═O)NR₂ group in which R is an aryl group as defined supra.

“Heterocyclylaminocarbonyl” represents a —C(═O)NHR or —C(═O)NR₂ group in which R is a heterocyclic group as defined supra. In certain embodiments, NR₂ is a heterocyclic ring, which is optionally substituted.

“Heteroarylaminocarbonyl” represents a —C(═O)NHR or —C(═O)NR₂ group in which R is a heteroaryl group as defined supra. In certain embodiments, NR₂ is a heteroaryl ring, which is optionally substituted.

“Cyano” represents a —CN moiety.

“Hydroxyl” represents a —OH moiety.

“Alkoxy” represents an —O-alkyl group in which the alkyl group is as defined supra. Examples include methoxy, ethoxy, n-propoxy, iso-propoxy, and the different butoxy, pentoxy, hexyloxy and higher isomers.

“Aryloxy” represents an —O-aryl group in which the aryl group is as defined supra. Examples include, without limitation, phenoxy and naphthoxy.

“Alkenyloxy” represents an —O-alkenyl group in which the alkenyl group is as defined supra. An example is allyloxy.

“Heterocyclyloxy” represents an —O-heterocyclyl group in which the heterocyclic group is as defined supra.

“Heteroaryloxy” represents an —O-heteroaryl group in which the heteroaryl group is as defined supra. An example is pyridyloxy.

“Alkanoate” represents an —OC(═O)—R group in which R is an alkyl group as defined supra.

“Aryloate” represents a —OC(═O)—R group in which R is an aryl group as defined supra.

“Heterocyclyloate” represents an —OC(═O)—R group in which R is a heterocyclic group as defined supra.

“Heteroaryloate” represents an —OC(═O)—R group in which P is a heteroaryl group as defined supra.

“Amino” represents an —NH₂ moiety.

“Alkylamino” represents an —NHR or —NR₂ group in which R is an alkyl group as defined supra. Examples include, without limitation, methylamino, ethylamino, n-propylamino, isopropylamino, and the different butylamino, pentylamino, hexylamino and higher isomers.

“Arylamino” represents an —NHR or —NR₂ group in which R is an aryl group as defined supra. An example is phenylamino.

“Heterocyclylamino” represents an —NHR or —NR₂ group in which R is a heterocyclic group as defined supra. In certain embodiments, NR₂ is a heterocyclic ring, which is optionally substituted.

“Heteroarylamino” represents a —NHR or —NR₂ group in which R is a heteroaryl group as defined supra. In certain embodiments, NR₂ is a heteroaryl ring, which is optionally substituted.

“Carbonylamino” represents a carboxylic acid amide group —NHC(═O)R that is linked to the rest of the molecule through a nitrogen atom.

“Alkylcarbonylamino” represents a —NHC(═O)R group in which R is an alkyl group as defined supra.

“Arylcarbonylamino” represents an —NHC(═O)R group in which R is an aryl group as defined supra.

“Heterocyclylcarbonylamino” represents an —NHC(═O)R group in which R is a heterocyclic group as defined supra.

“Heteroarylcarbonylamino” represents an —NHC(═O)R group in which R is a heteroaryl group as defined supra.

“Nitro” represents a —NO₂ moiety.

“Alkylthio” represents an —S-alkyl group in which the alkyl group is as defined supra. Examples include, without limitation, methylthio, ethylthio, n-propylthio, iso propylthio, and the different butylthio, pentylthio, hexylthio and higher isomers.

“Arylthio” represents an —S-aryl group in which the aryl group is as defined supra. Examples include phenylthio and naphthylthio.

“Heterocyclylthio” represents an —S-heterocyclyl group in which the heterocyclic group is as defined supra.

“Heteroarylthio” represents an —S-heteroaryl group in which the heteroaryl group is as defined supra.

“Sulfonyl” represents an —SO₂R group that is linked to the rest of the molecule through a sulfur atom.

“Alkylsulfonyl” represents an —SO₂-alkyl group in which the alkyl group is as defined supra.

“Arylsulfonyl” represents an —SO₂-aryl group in which the aryl group is as defined supra.

“Heterocyclylsulfonyl” represents an —SO₂-heterocyclyl group in which the heterocyclic group is as defined supra.

“Heteoarylsulfonyl” presents an —SO₂-heteroaryl group in which the heteroaryl group is as defined supra.

“Aldehyde” represents a —C(═O)H group.

“Alkanal” represents an alkyl-(C═O)H group in which the alkyl group is as defined supra.

“Alkylsilyl” presents an alkyl group that is linked to the rest of the molecule through the silicon atom, which may be substituted with up to three independently selected alkyl groups in which each alkyl group is as defined supra.

“Alkenylsilyl” presents an alkenyl group that is linked to the rest of the molecule through the silicon atom, which may be substituted with up to three independently selected alkenyl groups in which each alkenyl group is as defined supra.

“Alkynylsilyl” presents an alkynyl group that is linked to the rest of the molecule through the silicon atom, which may be substituted with up to three independently selected alkynyl groups in which each alkenyl group is as defined supra.

“Aryl” refers to a carbocyclic aromatic group. Examples of aryl groups include, but are not limited to, phenyl, naphthyl and anthracenyl. A carbocyclic aromatic group or a heterocyclic aromatic group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R¹, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

The term “C₁₋₁₀alkyl,” as used herein refers to a straight chain or branched, saturated or unsaturated hydrocarbon having from 1 to 10 carbon atoms. Representative “C₁₋₁₀alkyl” groups include, but are not limited to, -methyl, -ethyl, -n-propyl, -n-butyl, -n-pentyl, -n-hexyl, -n-heptyl, -n-octyl, -n-nonyl and -n-decyl; while branched C₁-C₈ alkyls include, but are not limited to, -isopropyl, -sec-butyl, -isobutyl, -tert-butyl, -isopentyl, 2-methylbutyl, unsaturated C₁-C₈ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, -3-methyl-1 butynyl, methyl, ethyl, propyl, isopropyl, n-butyl, isobutyl, sec-butyl, tert-butyl, n-pentyl, isopentyl, neopentyl, n-hexyl, isohexyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R¹, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

A “C₁₋₁₀alkylene” is a straight chain, saturated hydrocarbon group of the formula —(CH₂)₁₋₁₀—. Examples of a C₁-C₁₀ alkylene include methylene, ethylene, propylene, butylene, pentylene, hexylene, heptylene, ocytylene, nonylene and decalene.

An “arylene” is an aryl group which has two covalent bonds and can be in the ortho, meta, or para configurations as shown in the following structures:

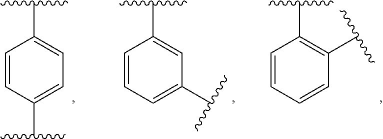 [SMILES: *c1ccc(*)cc1 ; *c1cccc(*)c1 ; *c1ccccc1*]

in which the phenyl group can be unsubstituted or substituted with up to four groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“Alkenylene” refers to an unsaturated, branched or straight chain or cyclic hydrocarbon radical of 2 to 18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2 to 18 carbon atoms, and having two monovalent radical centers derived by the removal of two hydrogen atoms from the same or two different carbon atoms of a parent alkyne. Typical alkynylene radicals include, but are not limited to: acetylene (—C≡C—), propargyl (—CH₂C≡C—), and 4-pentynyl (—CH₂CH₂CH₂C≡CH—).

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g., the alkyl moiety, including alkanyl, alkenyl or alkynyl groups, of the arylalkyl group is 1 to 6 carbon atoms and the aryl moiety is 5 to 14 carbon atoms.

“Heteroarylalkyl” refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g., the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from nitrogen, oxygen, phosphorus, and sulfur. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from nitrogen, oxygen, phosphorus, and sulfur), for example: a bicyclo [4,5], [5,5], [5,6], or [6,6] system.

“Substituted alkyl”, “substituted aryl”, and “substituted arylalkyl” mean alkyl, aryl, and arylalkyl respectively, in which one or more hydrogen atoms are each independently replaced with a substituent. Typical substituents include, but are not limited to, —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NR₂, —SO₃, —SO₃H, —S(═O)₂R, —OS(═O)₂OR, —S(═O)₂NR, —S(═O)R, —OP(═O)(OR)₂, —P(═O)(OR)₂, —PO⁻₃, —PO₃H₂, —C(═O)R, —C(═O)X, —C(═S)R, —CO₂R, —CO₂⁻, —C(═S)OR, —C(═O)SR, —C(═S)SR, —C(═O)NR₂, —C(═S)NR₂, —C(═NR)NR₂, where each X is independently a halogen: F, Cl, Br, or I; and each R is independently —H, C₂-C₂₀ alkyl, C₆-C₂₀ aryl, C₃-C₁₄ heterocycle, protecting group or prodrug moiety. Alkylene, alkenylene, and alkynylene groups as described above may also be similarly substituted.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

The phrase “pharmaceutically acceptable salt”, as used herein, refers to pharmaceutically acceptable organic or inorganic salts of an Exemplary Compound or Exemplary Conjugate. The Exemplary Compounds and Exemplary Conjugates contain at least one amino group, and accordingly acid addition salts can be formed with this amino group. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

“Pharmaceutically acceptable solvate” or “solvate” refer to an association of one or more solvent molecules and a compound of the invention, e.g., an Exemplary Compound or Exemplary Conjugate. Examples of solvents that form pharmaceutically acceptable solvates include, but are not limited to, water, isopropanol, ethanol, methanol, DMSO, ethyl acetate, acetic acid, and ethanolamine.

### Mononegavirales

The Mononegavirales order includes several families that further include numerous genera consisting of many different species.

The Mononegavirales order includes the Bornaviridae viruses, the Filoviridae viruses, the Mymonaviridae viruses, the Nyamiviridae viruses, the Paramyxoviridae viruses, the Pneumoviridae viruses, the Rhabdoviridae viruses, the Orthomyxoviridae viruses, and the Sunviridae viruses. Therefore, in some embodiments the viral infection belongs to the Bornaviridae family of viruses. In some embodiments, the viral infection belongs to the Filoviridae family of viruses. In some embodiments, the viral infection belongs to the Mymonaviridae family of viruses. In some embodiments, the viral infection belongs to the Nyamiviridae family of virus. In some embodiments, the viral infection belongs to the Paramyxoviridae family of viruses. In some embodiments, the viral infection belongs to the Pneumoviridae family of viruses. In some embodiments, the viral infection belongs to the Rhabdoviridae family of viruses. In some embodiments, the viral infection belongs to the Orthomyxoviridae viruses. In some embodiments, the viral infection belongs to the Sunviridae family of viruses.

The Bornaviridae family of viruses includes Loveridge's garter snake virus 1 (LGSV-1), Borna disease virus 1 (BoDV-1), Borna disease virus 2 (BoDV-2), variegated squirrel bornavirus 1 (VSBV-1), canary bornavirus 1 (CnBV-1), canary bornavirus 2 (CnBV-2), canary bornavirus 3 (CnBV-3), estrildid finch bornavirus 1 (EsBV-1), parrot bornavirus 1 (PaBV-1), parrot bornavirus 2 (PaBV-2), parrot bornavirus 3 (PaBV-3), parrot bornavirus 4 (PaBV-4), parrot bornavirus 7 (PaBV-7), parrot bornavirus 5 (PaBV-5), aquatic bird bornavirus 1 (ABBV-1), and aquatic bird bornavirus 2 (ABBV-2).

The Filoviridae family of viruses includes Lloviu virus (LLOV), Bundibugyo virus (BDBV), Reston virus (RESTV), Sudan virus (SUDV), Taï Forest virus (TAFV), Ebola virus (EBOV), Marburg virus (MARV), and Ravn virus (RAVV). In one example, the viral infection is Ebola virus (EBOV). In one example, the viral infection is Marburg virus (MARV).

The Mymonaviridae family of viruses includes Sclerotinia sclerotiorum negative-stranded RNA virus 1 (SsNSRV-1).

The Nyamiviridae family of viruses includes Midway virus (MIDWV), Nyamanini virus (NYMV), Sierra Nevada virus (SNVV), Pteromalus puparum negative-strand RNA virus 1 (PpNSRV-1), and soybean cyst nematode virus 1 (SbCNV-1).

The Paramyxoviridae family of viruses includes Atlantic salmon paramyxovirus (AsaPV), avian paramyxovirus 1 (APMV-1), avian paramyxovirus 2 (APMV-2), avian paramyxovirus 3 (APMV-3), avian paramyxovirus 4 (APMV-4), avian paramyxovirus 5 (APMV-5), avian paramyxovirus 6 (APMV-6), avian paramyxovirus 7 (APMV-7), avian paramyxovirus 8 (APMV-8), avian paramyxovirus 9 (APMV-9), avian paramyxovirus 10 (APMV-10), avian paramyxovirus 11 (APMV-11), avian paramyxovirus 12 (APMV-12), avian paramyxovirus 13 (APMV-13), Fer-de-Lance virus (FDLV), Cedar virus (CedV), Kumasi virus (KV), Hendra virus (HeV), Mòjiāng virus (MojV), Nipah virus (NiV), canine distemper virus (CDV), cetacean morbillivirus (CeMV), feline morbillivirus (FeMV), measles virus (MeV), peste-des-petits-ruminants virus (PPRV), phocine distemper virus (PDV), rinderpest virus (RPV), bovine parainfluenza virus 3 (BPIV-3), human parainfluenza virus 1 (HPIV-1), human parainfluenza virus 3 (HPIV-3), Sendai virus (SeV), porcine parainfluenza virus 1 (PPIV-1), Achimota virus 1 (AchPV-1), Achimota virus 2 (AchPV-2), bat mumps virus (BMV), parainfluenza virus 5 (PIV-5), human parainfluenza virus 1 (HPIV-1), human parainfluenza virus 2 (HPIV-2), human parainfluenza virus 4a (HPIV-4a), human parainfluenza virus 4b (HPIV-4b), Mapuera virus (MapV), Menangle virus (MenPV), mumps virus (MuV), La Piedad Michoacan Mexico virus (LPMV), Newcastle disease virus (NDV), simian virus 41 (SV-41), Sosuga virus, Teviot virus (TevPV), Tioman virus (TioPV), Tuhoko virus 1 (ThkPV-1), Tuhoko virus 2 (ThkPV-2), and Tuhoko virus 3 (ThkPV-3). In one example, the viral infection is measles virus (MeV). In one example, the viral infection is mumps virus (MuV). In one example, the viral infection is human parainfluenza virus 1 (HPIV-1). In one example, the viral infection is Hendra virus. In one example, the viral infection is Nipah virus. In one example, the viral infection is Newcastle disease virus (NDV). In one example, the viral infection is Sendai virus.

The Pneumoviridae family of viruses includes avian metapneumovirus (AMPV), human metapneumovirus (HMPV), bovine respiratory syncytial virus (BRSV), human respiratory syncytial virus (HRSV), human respiratory syncytial virus A2 (HRSV-A2), human respiratory syncytial virus B1 (HRSV-B1), and murine pneumonia virus (MPV). In one example, the viral infection is human respiratory syncytial virus (HRSV). In one example, the viral infection is human respiratory syncytial virus A2 (HRSV-A2). In one example, the viral infection is human respiratory syncytial virus B1 (HRSV-B1). In one example, the viral infection is human respiratory syncytial virus (HRSV). In one example, the viral infection is avian metapneumovirus (AMPV). In one example, the viral infection is human metapneumovirus (HMPV).

The Rhabdoviridae family of viruses includes Arboretum virus (ABTV), Balsa virus (BALV), Coot Bay virus (CBV), Puerto Almendras virus (PTAMV), Rio Chico virus (RCHV), Curionopolis virus (CURV), Iriri virus (IRIRV), Itacaiunas virus (ITAV), Rochambeau virus (RBUV), alfalfa dwarf virus (ADV), barley yellow striate mosaic virus (BYSMV), broccoli necrotic yellows virus (BNYV), Colocasia bobone disease-associated virus (CBDaV), festuca leaf streak virus (FLSV), lettuce necrotic yellows virus (LNYV), lettuce yellow mottle virus (LYMoV), northern cereal mosaic virus (NCMV), sonchus virus (SonV), strawberry crinkle virus (SCV), wheat American striate mosaic virus (WASMV), coffee ringspot virus (CoRSV), orchid fleck virus (OFV), Adelaide River virus (ARV), Berrimah virus (BRMV), bovine ephemeral fever virus (BEFV), Kimberley virus (KIMV), Malakal virus (MALV), Koolpinyah virus (KOOLV), kotonkan virus (KOTV), Obodhiang virus (OBOV), Yata virus (YATV), Flanders virus (FLAV), Hart Park virus (HPV), Gray Lodge virus (GLOV), Joinjakaka virus (JOIV), La Joya virus (LJV), Kamese virus (KAMV), Landjia virus (LANV=LJAV), Manitoba virus (MANV=MNTBV), Marco virus (MCOV), Mosqueiro virus (MQOV), Mossuril virus (MOSV), Ngaingan virus (NGAV), Ord River virus (ORV), Parry Creek virus (PCV), Wongabel virus (WONV), Barur virus (BARV), Fikirini virus (FKRV), Fukuoka virus (FUKV), Kern Canyon virus (KCV), Keuraliba virus (KEUV), Kolente virus (KOLEV), Kumasi rhabdovirus (KRV), Le Dantec virus (LDV), Mount Elgon bat virus (MEBV), Nkolbisson virus (NKOV), Nishimuro virus (NISV), Oita virus (OITAV), Wuhan louse fly virus 5 (WLFV-5), Y\check{o}ngjiā tick virus 2 (YTV-2), Aravan virus (ARAV), Australian bat lyssavirus (ABLV), Bokeloh bat lyssavirus (BBLV), Duvenhage virus (DUVV), European bat lyssavirus 1 (EBLV-1), European bat lyssavirus 2 (EBLV-2), Ikoma lyssavirus (IKOV), Irkut virus (IRKV), Khujand virus (KHUV), Lagos bat virus (LBV), Mokola virus (MOKV), rabies virus (RABV), Shimoni bat virus (SHIBV), West Caucasian bat virus (WCBV), Hirame rhabdovirus (HIRV), viral hemorrhagic septicemia virus (VHSV), infectious hematopoietic necrosis virus (IHNV), snakehead rhabdovirus (SHRV), datura yellow vein virus (DYVV), eggplant mottled dwarf virus (EMDV), maize fine streak virus (MSFV), maize Iranian mosaic virus (MIMV), maize mosaic virus (MMV), potato yellow dwarf virus (PYDV), rice yellow stunt virus (RYSV), rice transitory yellowing virus (RTYV), sonchus yellow net virus (SYNV), sowthistle yellow vein virus (SYVV), taro vein chlorosis virus (TaVCV), eel virus European X (EVEX), perch rhabdovirus (PRV), lake trout rhabdovirus (LTRV), Drosophila affinis sigmavirus (DAffSV), Drosophila ananassae sigmavirus (DAnaSV), Drosophila immigrans sigmavirus (DImmSV), Drosophila melanogaster sigmavirus (DMelSV), Drosophila obscura sigmavirus (DObsSV), Drosophila tristis sigmavirus (DTriSV), Muscina stabulans sigmavirus (MStaSV), spring viremia of carp virus (SVCV), grass carp rhabdovirus (GrCRV), pike fry rhabdovirus (PFRV), tench rhabdovirus (TenRV), Bas-Congo virus (BASV), Coastal Plains virus (CPV), Ekpoma virus 1 (EKV-1), Ekpoma virus 2 (EKV-2), Sweetwater Branch virus (SWBV), Bivens Arm virus (BAV), Tibrogargan virus (TIBV), Durham virus (DURV), Klamath virus (KLAV), tupaia virus (TUPV), lettuce big-vein associated virus (LBVaV), vesicular stomatitis Alagoas virus (VSAV), American bat vesiculovirus (ABVV), Carajás virus (CJSV), Chandipura virus (CHPV), Cocal virus (COCV), vesicular stomatitis Indiana virus (VSIV), Isfahan virus (ISFV), Jurona virus (JURV), Malpais Spring virus (MSPV), Maraba virus (MARAV), Morreton virus (MORV), vesicular stomatitis New Jersey virus (VSNJV), Perinet virus (PERV), Piry virus (PIRYV), Radi virus (RADV), Yug Bogdanovac virus (YBV), and Moussa virus (MOUV). In one example, the viral infection is bovine ephemeral fever virus (BEFV). In one example, the viral infection is maize mosaic virus (MMV). In one example, the viral infection is rice yellow stunt virus (RYSV). In one example, the viral infection is lettuce necrotic yellows virus (LNYV). In one example, the viral infection is Rabies virus (RABV). In one example, the viral infection is infectious hematopoietic necrosis virus (IHNV). In one example, the viral infection is vesicular stomatitis Indiana virus (VSIV).

The Orthomyxoviridae family of viruses includes Influenza virus A, Influenza virus B, Influenza virus C, Influenza virus D, Isavirus, Thogotovirus, and Quaranjavirus. In one example, the viral infection is Influenza virus A.

The Sunviridae family of viruses includes Sunshine Coast virus (SunCV).

### Angiotensin II Signalling Inhibitor

The angiotensin II signalling pathway, which is also known as the renin-angiotensin system (RAS) or renin-angiotensin-aldosterone system (RAAS), is a hormone system that is involved in the regulation of plasma sodium concentration and arterial blood pressure. When plasma sodium concentration is lower than normal or renal blood flow is reduced, the juxtaglomerular cells in the kidneys convert prorenin (an intracellular protein) into renin, which is then secreted directly into the circulatory system. This renin then cleaves angiotensin I from a plasma protein known as angiotensinogen. Angiotensin I is then converted to angiotensin II by the angiotensin-converting enzyme (ACE), which is found in the endothelial cells of the capillaries throughout the body, within the lungs and the epithelial cells of the kidneys. Angiotensin II is a potent vaso-active peptide that causes the arterioles to constrict, resulting in increased arterial blood pressure. Angiotensin II also stimulates the secretion of the hormone aldosterone from the adrenal cortex. Aldosterone causes the tubular epithelial cells of the kidneys to increase the reabsorption of sodium ions from the tubular fluid back into the blood, while at the same time causing them to excrete potassium ions into the tubular fluid which will become urine.

Angiotensin II is the endogenous ligand for the angiotensin receptor. The angiotensin receptor belongs to the class of G protein-coupled receptors (GPCRs) that are responsible for signal transduction in the angiotensin II signalling pathway. The term “receptor”, as used to herein, refers to a protein molecule that receives a chemical signal and produces a biological response. In one example, the receptor is the angiotensin receptor.

Two subtypes of the angiotensin receptor are known to exist—the angiotensin I receptor and the angiotensin II receptor. Four subtypes of the angiotensin II receptor are known to exist—the angiotensin II receptor type 1 (AT₁), the angiotensin II receptor type 2 (AT₂), the angiotensin II receptor type 3 (AT₃), and the angiotensin II receptor type 4 (AT₄).

There are molecules which are known to interrupt, or inhibit, the angiotensin II signalling pathway. As used herein, the term “angiotensin II signalling inhibitor” refers to an agent capable of interrupting or inhibiting the angiotensin II signalling pathway. Three modes by which angiotensin II signalling may be inhibited are; (1) by antagonism of the angiotensin II receptor, (2) by inhibition of angiotensin-converting enzyme (ACE), and (3) by inhibition of renin. As used herein, the term “antagonising” or “antagonism” refers to the blocking or dampening of a biological response by binding to the receptor. The term “inhibiting” or “inhibition,” as used herein, refers to any detectable negative effect on a target biological process, such as cellular signal transduction, cell proliferation, viral replication, and viral infection. Typically, an antagonism or inhibition is reflected in a decrease of at least 10%, 20%, 30%, 40%, or 50% in target process (e.g., angiotensin II signalling or viral infection), or any one of the downstream parameters mentioned above, when compared to a control. In one example, the angiotensin II signalling pathway is interrupted or inhibited by antagonism of the angiotensin II receptor (i.e., by an angiotensin II receptor antagonist). In one example, the angiotensin II signalling pathway is interrupted or inhibited by inhibition of angiotensin-converting enzyme (ACE) (i.e., by an ACE inhibitor). In one example, the angiotensin II signalling pathway is interrupted or inhibited by inhibition of renin (i.e., by a renin inhibitor).

Such drugs which are known to interrupt, or inhibit, signalling of the angiotensin II signalling pathway are often small molecules. As used herein, the term “small molecule” refers to an organic molecule with a molecular weight of generally less than 900 Daltons. Larger molecules such as, for example, nucleic acids, proteins and polysaccharides, are not considered small molecules. The person skilled in the art would appreciate that organic small molecules are particularly useful as therapeutic agents. In one example, the angiotensin II signalling inhibitor is a small molecule. In one example, the angiotensin II receptor antagonist is a small molecule. In one example, the angiotensin-converting enzyme (ACE) inhibitor is a small molecule. In one example, the renin inhibitor is a small molecule.

In an alternate embodiment, the angiotensin II signalling inhibitor is an antibody. For example, an antibody which binds the angiotensin II receptor.

### Angiotensin II Receptor Antagonists

In some embodiments, the angiotensin II signalling inhibitor is an angiotensin II receptor antagonist. As used herein, the term “angiotensin II receptor antagonist” refers to a molecule that blocks the angiotensin II receptor. Angiotensin II receptor antagonists are also referred to as angiotensin receptor blockers (ARBs). An angiotensin II receptor antagonist blocks the activation of the angiotensin II receptor, thereby preventing angiotensin II from binding, and consequently blocking the biological activity of the angiotensin II receptor. In one example, the angiotensin II signalling inhibitor is an angiotensin II receptor type 1 (AT₁) antagonist.

In some embodiments, the angiotensin II signalling inhibitor has a structure according to Formula I:

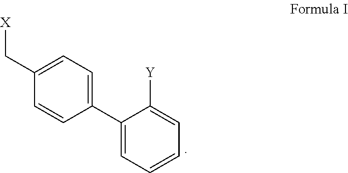

The above compounds of Formula I may be further described as follows.

According to a compound of Formula I, X is selected from the group consisting of:

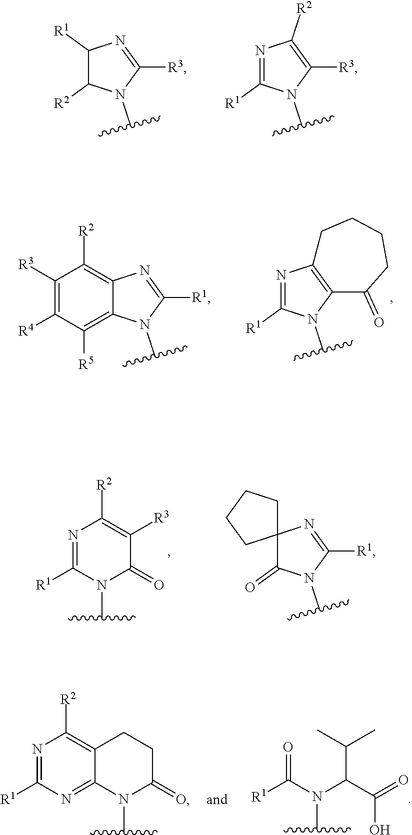

In one example, the angiotensin II signalling inhibitor according to Formula I is

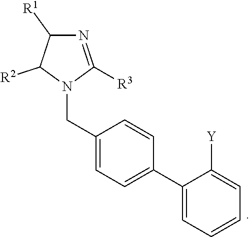

In one example, the angiotensin II signalling inhibitor according to Formula I is

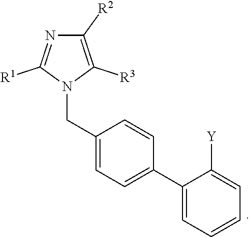

In one example, the angiotensin II signalling inhibitor according to Formula I is

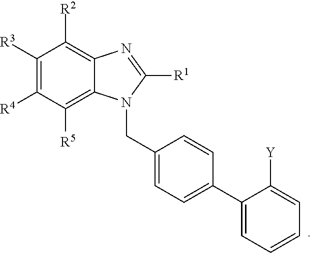

In one example, the angiotensin II signalling inhibitor according to Formula I is

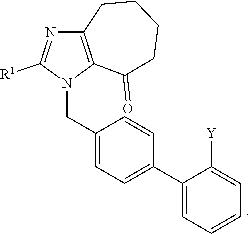

In one example, the angiotensin II signalling inhibitor according to Formula I is

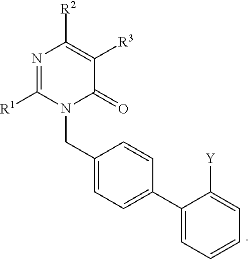

In one example, the angiotensin II signalling inhibitor according to Formula I is

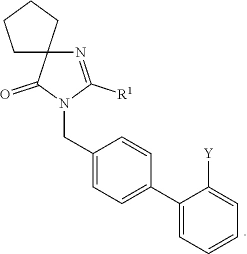

In one example, the angiotensin II signalling inhibitor according to Formula I is

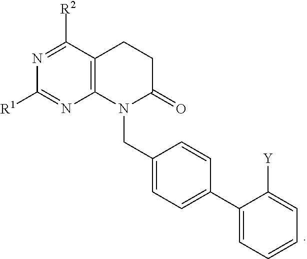

In one example, the angiotensin II signalling inhibitor according to Formula I is

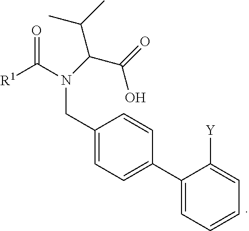

According to a compound of Formula I, Y is selected from the group consisting of:

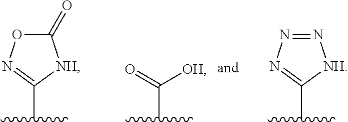

In one example, the angiotensin II signalling inhibitor according to Formula I is

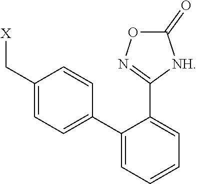

In one example, the angiotensin II signalling inhibitor according to Formula I is

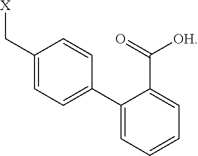

In one example, the angiotensin II signalling inhibitor according to Formula I is

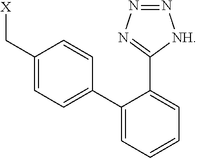

It will be understood that the angiotensin II signalling inhibitor according to Formula I may be any combination of X and Y groups.

In one example, the angiotensin II signalling inhibitor according to Formula I is

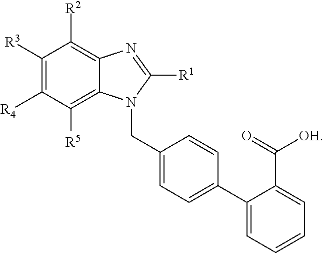

In one example, the angiotensin II signalling inhibitor according to Formula I is

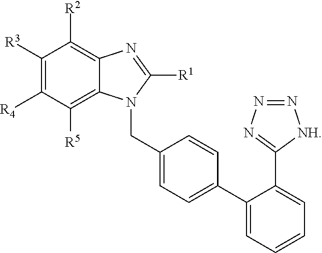

In one example, the angiotensin II signalling inhibitor according to Formula I is

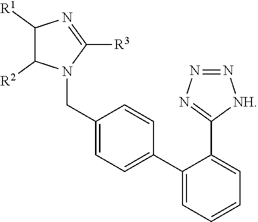

According to a compound of Formula I, R¹, R², R³, R⁴ and R⁵ are each independently selected from the group consisting of hydrogen, halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, monocyclic or polycyclic carbocyclic, and monocyclic or polycyclic heterocyclic.

In one example, the angiotensin II signalling inhibitor according to Formula I is

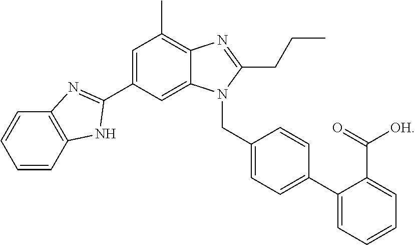 [SMILES: CCCc1nc2c(C)cc(-c3nc4ccccc4[nH]3)cc2n1Cc1ccc(-c2ccccc2C(=O)O)cc1]

In one example, the angiotensin II signalling inhibitor according to Formula I is

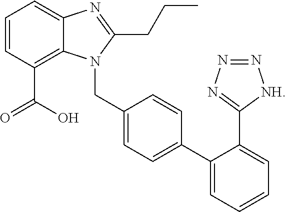 [SMILES: CCCc1nc2cccc(C(=O)O)c2n1Cc1ccc(-c2ccccc2-c2nnn[nH]2)cc1]

The C₁₋₁₀alkyl, C₂₋₁₀alkenyl, monocyclic or polycyclic carbocyclic, and monocyclic or polycyclic heterocyclic are each optionally substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, monocyclic or polycyclic carbocyclic, and monocyclic or polycyclic heterocyclic.

In one example, the angiotensin II signalling inhibitor according to Formula I is

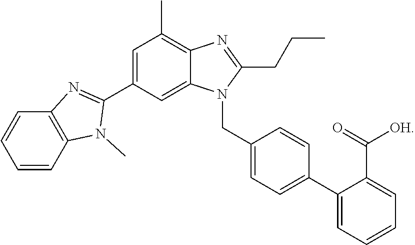 [SMILES: CCCc1nc2c(C)cc(-c3nc4ccccc4n3C)cc2n1Cc1ccc(-c2ccccc2C(=O)O)cc1]

In one example, the angiotensin II signalling inhibitor according to Formula I is

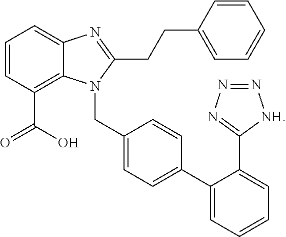 [SMILES: O=C(O)c1cccc2nc(CCc3ccccc3)n(Cc3ccc(-c4ccccc4-c4nnn[nH]4)cc3)c12]

The monocyclic or polycyclic carbocyclic, and monocyclic or polycyclic heterocyclic are each optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl.

In one example, the angiotensin II signalling inhibitor according to Formula I is

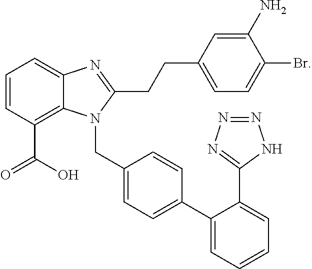 [SMILES: Nc1cc(CCc2nc3cccc(C(=O)O)c3n2Cc2ccc(-c3ccccc3-c3nnn[nH]3)cc2)ccc1Br]

Further, according to a compound of Formula I, the C₁₋₁₀alkyl and C₂₋₂₀alkenyl, wherever each appears within Formula I, may each be optionally interrupted with one or more heteroatoms independently selected from O, N and S.

In one example, the angiotensin II signalling inhibitor according to Formula I is

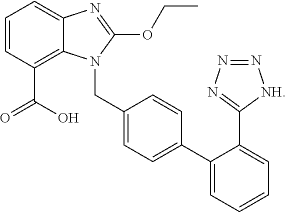 [SMILES: CCOc1nc2cccc(C(=O)O)c2n1Cc1ccc(-c2ccccc2-c2nnn[nH]2)cc1]

In one example, the angiotensin II signalling inhibitor according to Formula I is Telmisartan. In one example, the angiotensin II signalling inhibitor according to Formula I is Candesartan. In one example, the angiotensin II signalling inhibitor according to Formula I is Losartan. In one example, the angiotensin II signalling inhibitor according to Formula I is Irbesartan. In one example, the angiotensin II signalling inhibitor according to Formula I is Olmesartan. In one example, the angiotensin II signalling inhibitor according to Formula I is Azilsartan. In one example, the angiotensin II signalling inhibitor according to Formula I is Fimasartan. In one example, the angiotensin II signalling inhibitor according to Formula I is EXP-3174. In one example, the angiotensin II signalling inhibitor according to Formula I is Pratosartan. In one example, the angiotensin II signalling inhibitor according to Formula I is TCV-116. In one example, the angiotensin II signalling inhibitor according to Formula I is Tasosartan.

In some embodiments, the angiotensin II signalling inhibitor has a structure according to Formula II:

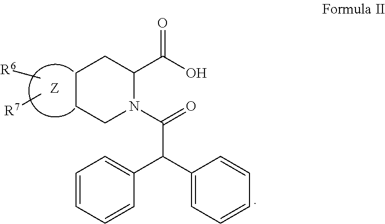

The above compounds of Formula II may be further described as follows.

According to a compound of Formula II, Z is a 5- or 6-membered monocyclic carbocyclic or monocyclic heterocyclic.

In one example, the angiotensin II signalling inhibitor according to Formula II is

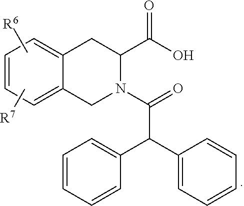

In one example, the angiotensin II signalling inhibitor according to Formula II is

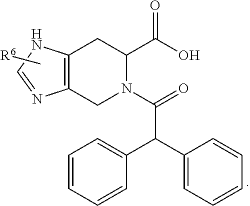

It will be appreciated that it is not always possible to have two substituents, i.e., both R⁶ and R⁷, on the Z group. However, it will be understood that the Z group of Formula II has a maximum of two substituents, i.e., both R⁶ and R⁷.

According to a compound of Formula II, R⁶ and R⁷ are each independently selected from the group consisting of hydrogen, halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, monocyclic or polycyclic carbocyclic, and monocyclic or polycyclic heterocyclic.

In one example, the angiotensin II signalling inhibitor according to Formula II is

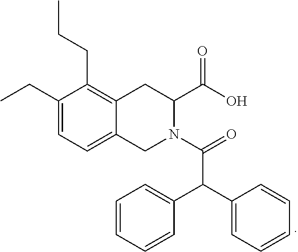 [SMILES: CCCc1c(CC)ccc2c1CC(C(=O)O)N(C(=O)C(c1ccccc1)c1ccccc1)C2]

In one example, the angiotensin II signalling inhibitor according to Formula II is

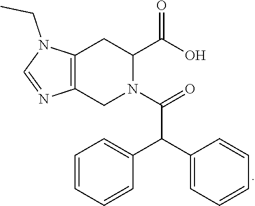 [SMILES: CCn1cnc2c1CC(C(=O)O)N(C(=O)C(c1ccccc1)c1ccccc1)C2]

The C₁₋₁₀alkyl, C₂₋₁₀alkenyl, monocyclic or polycyclic carbocyclic, and monocyclic or polycyclic heterocyclic are each optionally substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, monocyclic or polycyclic carbocyclic, and monocyclic or polycyclic heterocyclic.

In one example, the angiotensin II signalling inhibitor according to Formula II is

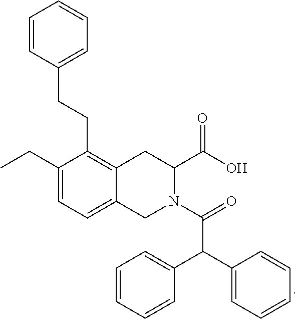 [SMILES: CCc1ccc2c(c1CCc1ccccc1)CC(C(=O)O)N(C(=O)C(c1ccccc1)c1ccccc1)C2]

In one example, the angiotensin II signalling inhibitor according to Formula II is

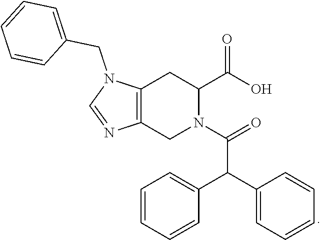 [SMILES: O=C(O)C1Cc2c(ncn2Cc2ccccc2)CN1C(=O)C(c1ccccc1)c1ccccc1]

The monocyclic or polycyclic carbocyclic, and monocyclic or polycyclic heterocyclic are each optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl.

In one example, the angiotensin II signalling inhibitor according to Formula II is

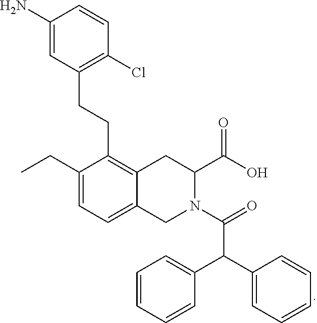 [SMILES: CCc1ccc2c(c1CCc1cc(N)ccc1Cl)CC(C(=O)O)N(C(=O)C(c1ccccc1)c1ccccc1)C2]

In one example, the angiotensin II signalling inhibitor according to Formula II is

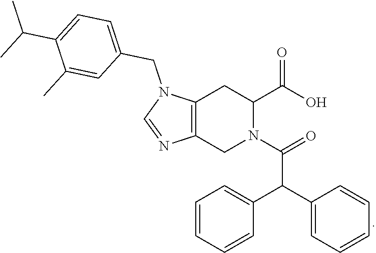 [SMILES: Cc1cc(Cn2cnc3c2CC(C(=O)O)N(C(=O)C(c2ccccc2)c2ccccc2)C3)ccc1C(C)C]

Further, according to a compound of Formula II, the C₁₋₁₀alkyl and C₂₋₂₀alkenyl, wherever each appears within Formula II, may each be optionally interrupted with one or more heteroatoms independently selected from O, N and S.

In one example, the angiotensin II signalling inhibitor according to Formula II is

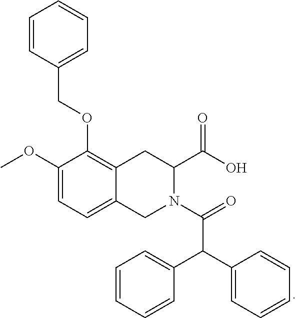 [SMILES: COc1ccc2c(c1OCc1ccccc1)CC(C(=O)O)N(C(=O)C(c1ccccc1)c1ccccc1)C2]

In one example, the angiotensin II signalling inhibitor according to Formula II is

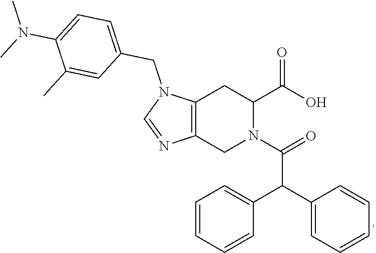 [SMILES: Cc1cc(Cn2cnc3c2CC(C(=O)O)N(C(=O)C(c2ccccc2)c2ccccc2)C3)ccc1N(C)C]

In one example, the angiotensin II signalling inhibitor according to Formula II is EMA401. In one example, the angiotensin II signalling inhibitor according to Formula II is PD123319.

In some embodiments, the angiotensin II signalling inhibitor has a structure according to Formula III:

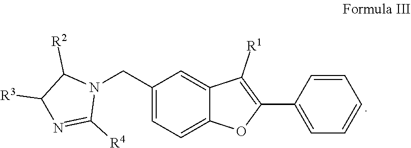

The above compounds of Formula III may be further described as follows.

According to a compound of Formula III, R¹, R², R³ and R⁴ are each independently selected from the group consisting of hydrogen, halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, monocyclic or polycyclic carbocyclic, and monocyclic or polycyclic heterocyclic.

In one example, the angiotensin II signalling inhibitor according to Formula III is

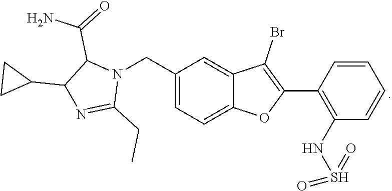

The C₁₋₁₀alkyl, C₂₋₁₀alkenyl, monocyclic or polycyclic carbocyclic, and monocyclic or polycyclic heterocyclic are each optionally substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, monocyclic or polycyclic carbocyclic, and monocyclic or polycyclic heterocyclic.

In one example, the angiotensin II signalling inhibitor according to Formula III is

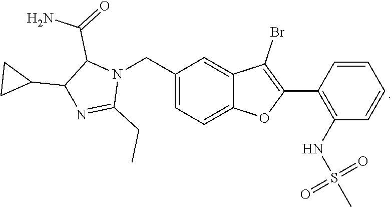 [SMILES: CCC1=NC(C2CC2)C(C(N)=O)N1Cc1ccc2oc(-c3ccccc3NS(C)(=O)=O)c(Br)c2c1]

The monocyclic or polycyclic carbocyclic, and monocyclic or polycyclic heterocyclic are each optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl. In one example, the angiotensin II signalling inhibitor according to Formula III is

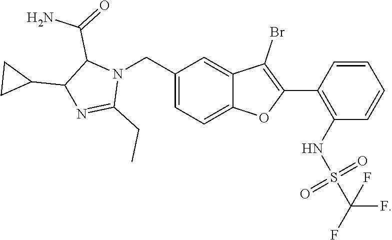 [SMILES: CCC1=NC(C2CC2)C(C(N)=O)N1Cc1ccc2oc(-c3ccccc3NS(=O)(=O)C(F)(F)F)c(Br)c2c1]

Further, according to a compound of Formula III, the C₁₋₁₀alkyl and C₂₋₂₀alkenyl, wherever each appears within Formula III, may each be optionally interrupted with one or more heteroatoms independently selected from O, N and S.

In one example, the angiotensin II signalling inhibitor according to Formula III is

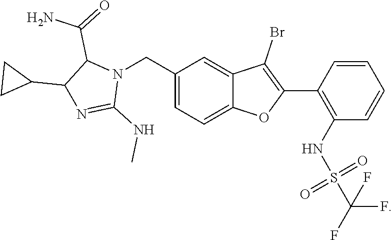 [SMILES: CNC1=NC(C2CC2)C(C(N)=O)N1Cc1ccc2oc(-c3ccccc3NS(=O)(=O)C(F)(F)F)c(Br)c2c1]

In one example, the angiotensin II signalling inhibitor according to Formula III is Saprisartan.

In some embodiments, the angiotensin II signalling inhibitor has a structure according to Formula IV:

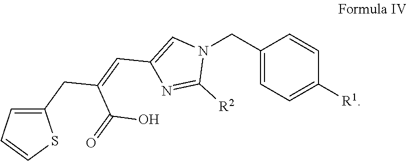

The above compounds of Formula IV may be further described as follows.

According to a compound of Formula IV, R¹ and R² are each independently selected from the group consisting of hydrogen, halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, monocyclic or polycyclic carbocyclic, and monocyclic or polycyclic heterocyclic.

In one example, the angiotensin II signalling inhibitor according to Formula IV is

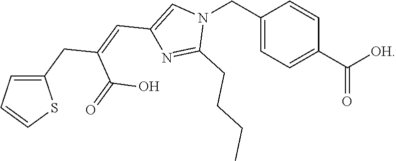 [SMILES: CCCCc1nc(/C=C(/Cc2cccs2)C(=O)O)cn1Cc1ccc(C(=O)O)cc1]

The C₁₋₁₀alkyl, C₂₋₁₀alkenyl, monocyclic or polycyclic carbocyclic, and monocyclic or polycyclic heterocyclic are each optionally substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, monocyclic or polycyclic carbocyclic, and monocyclic or polycyclic heterocyclic.

In one example, the angiotensin II signalling inhibitor according to Formula IV is

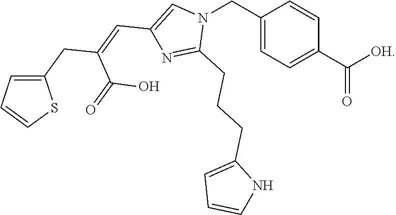 [SMILES: O=C(O)/C(=C\c1cn(Cc2ccc(C(=O)O)cc2)c(CCCc2ccc[nH]2)n1)Cc1cccs1]

The monocyclic or polycyclic carbocyclic, and monocyclic or polycyclic heterocyclic are each optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl.

In one example, the angiotensin II signalling inhibitor according to Formula IV is

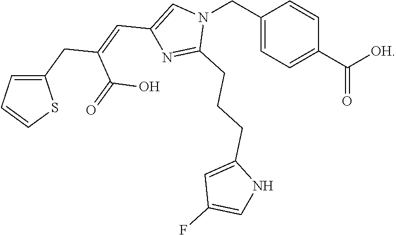 [SMILES: O=C(O)/C(=C\c1cn(Cc2ccc(C(=O)O)cc2)c(CCCc2cc(F)c[nH]2)n1)Cc1cccs1]

Further, according to a compound of Formula IV, the C₁₋₁₀alkyl and C₂₋₂₀alkenyl, wherever each appears within Formula IV, may each be optionally interrupted with one or more heteroatoms independently selected from O, N and S. In one example, the angiotensin II signalling inhibitor according to Formula IV is

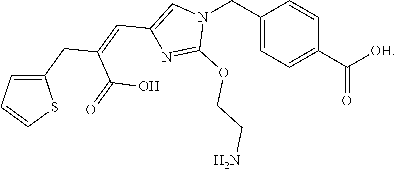 [SMILES: NCCOc1nc(/C=C(/Cc2cccs2)C(=O)O)cn1Cc1ccc(C(=O)O)cc1]

In one example, the angiotensin II signalling inhibitor according to Formula IV is Eprosartan.

It will be appreciated that any of the optional heteroatoms or substituents referred to above in Formula I, II, III, or IV, with reference to “one or more”, unless otherwise stated, may be any integer such as 1, 2, 3, 4, 5, 6, etc., or for example a range of 1 to 6 substituents, 1 to 3 substituents, or 1 to 2 substituents.

A number of small molecule angiotensin II receptor antagonists, specifically angiotensin AT₁ antagonists, are known to exist, and there are examples where AT₁ antagonists have reached the market. Examples include, but are not limited to, Losartan (trade name Cozaar), Candesartan (Atacand), Valsartan (Diovan/Exforge), Irbesartan (Avapro), Telmisartan (Micardis), Eprosartan (Teventen), Olmesartan (Benicar/Olmetec), Azilsartan (Edarbi), and Fimasartan (Kanarb). Other examples include, but are not limited to, Saprisartan, EXP-3174, Pratosartan, EMA401, TCV-116, PD123319, and Tasosartan (Table 1). In one example, the angiotensin II receptor antagonist is Losartan. In one example, the angiotensin II receptor antagonist is Candesartan. In one example, the angiotensin II receptor antagonist is Valsartan. In one example, the angiotensin II receptor antagonist is Irbesartan. In one example, the angiotensin II receptor antagonist is Telmisartan. In one example, the angiotensin II receptor antagonist is Eprosartan. In one example, the angiotensin II receptor antagonist is Olmestartan. In one example, the angiotensin II receptor antagonist is Azilsartan. In one example, the angiotensin II receptor antagonist is Fimasartan. In one example, the angiotensin II receptor antagonist is Saprisartan. In one example, the angiotensin II receptor antagonist is EXP-3174. In one example, the angiotensin II receptor antagonist is Pratosartan. In one example, the angiotensin II receptor antagonist is EMA401. In one example, the angiotensin II receptor antagonist is TCV-116. In one example, the angiotensin II receptor antagonist is PD123319. In one example, the angiotensin II receptor antagonist is Tasosartan.

TABLE 1
Structures of Known Angiotensin II Receptor Antagonists

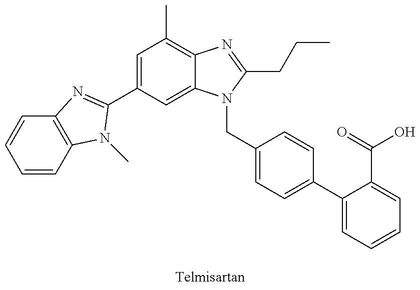 [SMILES: CCCc1nc2c(C)cc(-c3nc4ccccc4n3C)cc2n1Cc1ccc(-c2ccccc2C(=O)O)cc1]

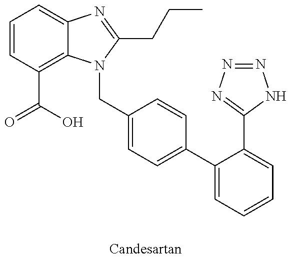 [SMILES: CCCc1nc2cccc(C(=O)O)c2n1Cc1ccc(-c2ccccc2-c2nnn[nH]2)cc1]

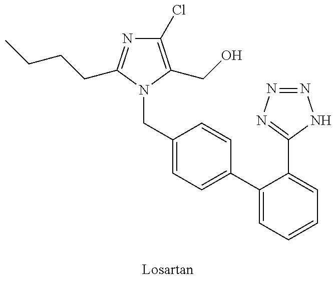 [SMILES: CCCCc1nc(Cl)c(CO)n1Cc1ccc(-c2ccccc2-c2nnn[nH]2)cc1]

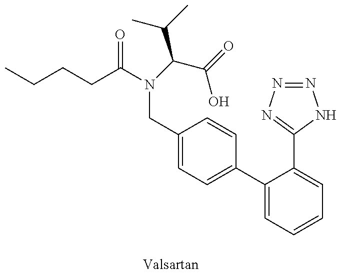 [SMILES: CCCCC(=O)N(Cc1ccc(-c2ccccc2-c2nnn[nH]2)cc1)[C@H](C(=O)O)C(C)C]

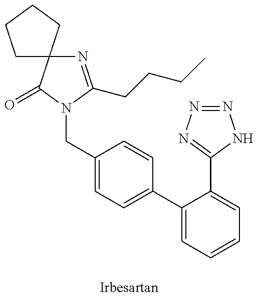 [SMILES: CCCCC1=NC2(CCCC2)C(=O)N1Cc1ccc(-c2ccccc2-c2nnn[nH]2)cc1]

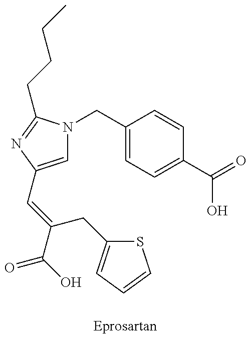 [SMILES: CCCCc1nc(/C=C(\Cc2cccs2)C(=O)O)cn1Cc1ccc(C(=O)O)cc1]

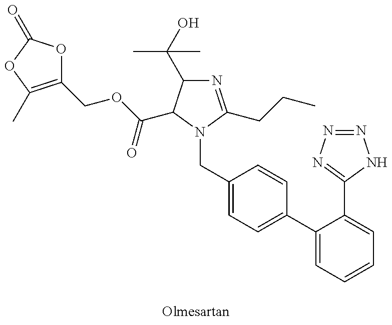 [SMILES: CCCC1=NC(C(C)(C)O)C(C(=O)OCc2oc(=O)oc2C)N1Cc1ccc(-c2ccccc2-c2nnn[nH]2)cc1]

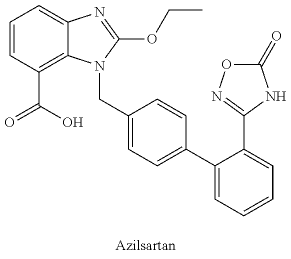 [SMILES: CCOc1nc2cccc(C(=O)O)c2n1Cc1ccc(-c2ccccc2-c2noc(=O)[nH]2)cc1]

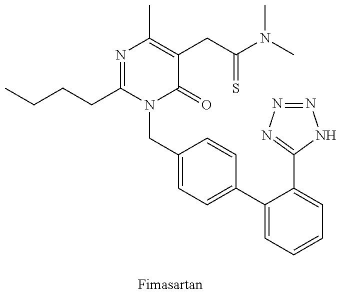 [SMILES: CCCCc1nc(C)c(CC(=S)N(C)C)c(=O)n1Cc1ccc(-c2ccccc2-c2nnn[nH]2)cc1]

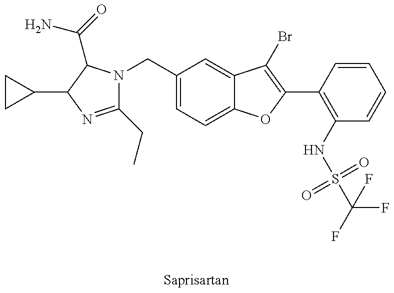 [SMILES: CCC1=NC(C2CC2)C(C(N)=O)N1Cc1ccc2oc(-c3ccccc3NS(=O)(=O)C(F)(F)F)c(Br)c2c1]

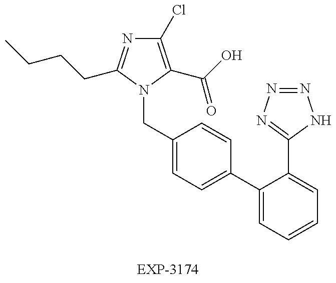 [SMILES: CCCCc1nc(Cl)c(C(=O)O)n1Cc1ccc(-c2ccccc2-c2nnn[nH]2)cc1]

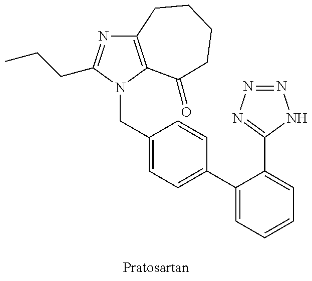 [SMILES: CCCc1nc2c(n1Cc1ccc(-c3ccccc3-c3nnn[nH]3)cc1)C(=O)CCCC2]

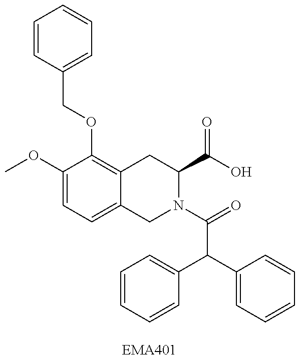 [SMILES: COc1ccc2c(c1OCc1ccccc1)C[C@@H](C(=O)O)N(C(=O)C(c1ccccc1)c1ccccc1)C2]

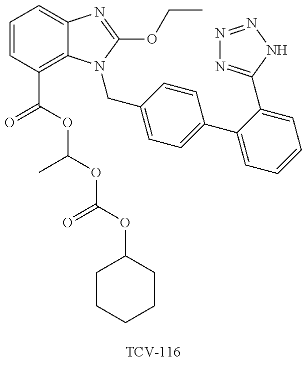 [SMILES: CCOc1nc2cccc(C(=O)OC(C)OC(=O)OC3CCCCC3)c2n1Cc1ccc(-c2ccccc2-c2nnn[nH]2)cc1]

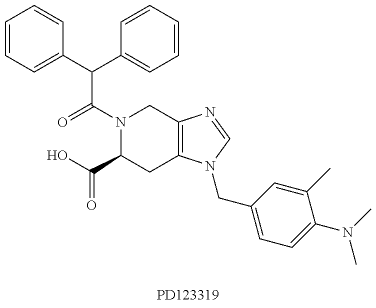 [SMILES: Cc1cc(Cn2cnc3c2C[C@@H](C(=O)O)N(C(=O)C(c2ccccc2)c2ccccc2)C3)ccc1N(C)C]

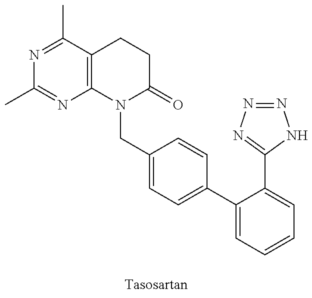 [SMILES: Cc1nc(C)c2c(n1)N(Cc1ccc(-c3ccccc3-c3nnn[nH]3)cc1)C(=O)CC2]

### Angiotensin-Converting Enzyme (ACE) Inhibitors

In some embodiments, the angiotensin II signalling inhibitor is an angiotensin-converting enzyme (ACE) inhibitor. As used herein, the term “angiotensin-converting enzyme inhibitor” refers to a molecule that inhibits the biological activity of the angiotensin-converting enzyme (ACE). Angiotensin-converting enzyme (ACE) is responsible for the conversion of angiotensin I to the biologically active angiotensin II.

In some embodiments, the angiotensin II signalling inhibitor has a structure according to Formula V:

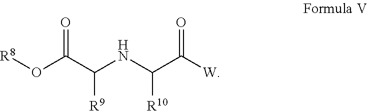

The above compounds of Formula V may be further described as follows.

According to a compound of Formula V, W is selected from the group consisting of:

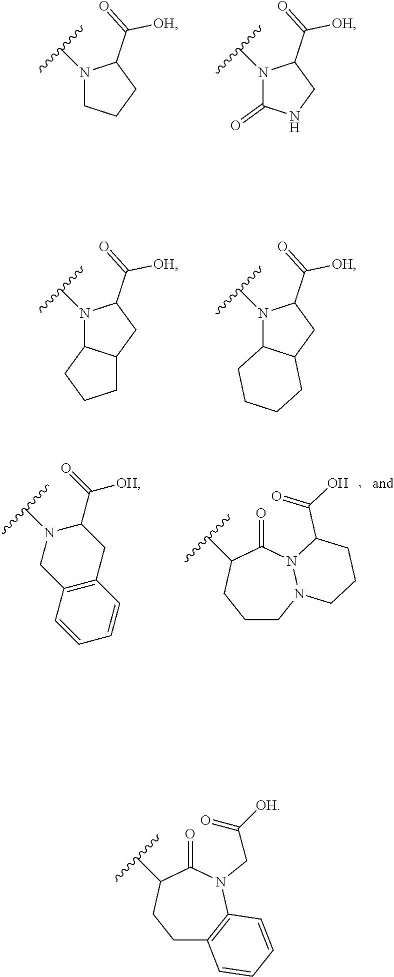

In one example, the angiotensin II signalling inhibitor according to Formula V is

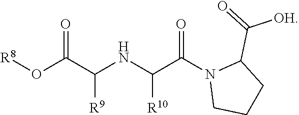

In one example, the angiotensin II signalling inhibitor according to Formula V is

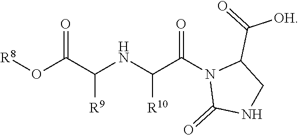

In one example, the angiotensin II signalling inhibitor according to Formula V is

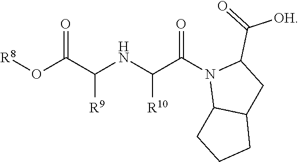

In one example, the angiotensin II signalling inhibitor according to Formula V is

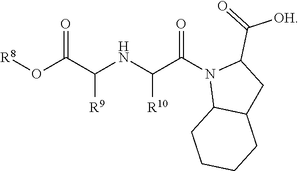

In one example, the angiotensin II signalling inhibitor according to Formula V is

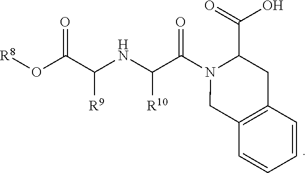

In one example, the angiotensin II signalling inhibitor according to Formula V is

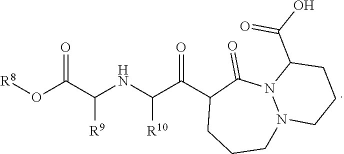

In one example, the angiotensin II signalling inhibitor according to Formula V is

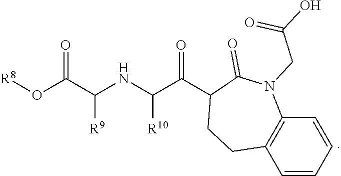

According to a compound of Formula V, W may be optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, C₁₋₁₀alkylaryl, C₁₋₁₀alkylC₁₋₁₀ cyclyl, monocyclic or polycyclic carbocyclic, and monocyclic or polycyclic heterocyclic.

In one example, the angiotensin II signalling inhibitor according to Formula V is

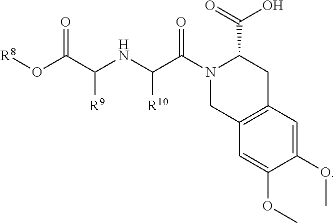

According to a compound of Formula V, R⁸, R⁹ and R¹⁰ are each independently selected from the group consisting of hydrogen, halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, C₁₋₁₀alkylaryl, monocyclic or polycyclic carbocyclic, and monocyclic or polycyclic heterocyclic.

In one example, the angiotensin II signalling inhibitor according to Formula V is

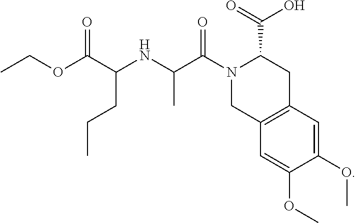 [SMILES: CCCC(NC(C)C(=O)N1Cc2cc(OC)c(OC)cc2C[C@H]1C(=O)O)C(=O)OCC]

The C₁₋₁₀alkyl, C₂₋₁₀alkenyl, monocyclic or polycyclic carbocyclic, and monocyclic or polycyclic heterocyclic are each optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, and aminocarbonyl.

In one example, the angiotensin II signalling inhibitor according to Formula V is

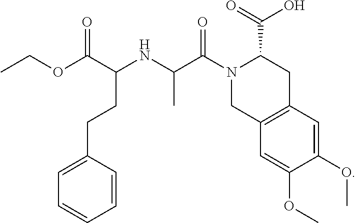 [SMILES: CCOC(=O)C(CCc1ccccc1)NC(C)C(=O)N1Cc2cc(OC)c(OC)cc2C[C@H]1C(=O)O]

Further, according to a compound of Formula V, the C₁₋₁₀alkyl, C₂₋₂₀alkenyl, and C₁₋₁₀alkylaryl, wherever each appears within Formula V, may each be optionally interrupted with one or more heteroatoms independently selected from O, N and S.

In one example, the angiotensin II signalling inhibitor according to Formula V is

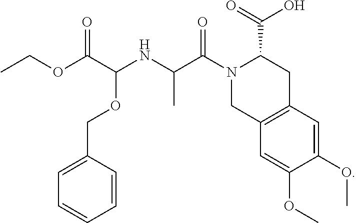 [SMILES: CCOC(=O)C(NC(C)C(=O)N1Cc2cc(OC)c(OC)cc2C[C@H]1C(=O)O)OCc1ccccc1]

In one example, the angiotensin II signalling inhibitor according to Formula V is Quinapril. In one example, the angiotensin II signalling inhibitor according to Formula V is Imidapril. In one example, the angiotensin II signalling inhibitor according to Formula V is Enalapril. In one example, the angiotensin II signalling inhibitor according to Formula V is Ramipril. In one example, the angiotensin II signalling inhibitor according to Formula V is Perindopril. In one example, the angiotensin II signalling inhibitor according to Formula V is Trandolapril. In one example, the angiotensin II signalling inhibitor according to Formula V is Lisinopril. In one example, the angiotensin II signalling inhibitor according to Formula V is Moexipril.

In some embodiments, the angiotensin II signalling inhibitor has a structure according to Formula VI:

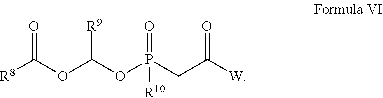

The above compounds of Formula VI may be further described as follows.

According to a compound of Formula VI, W is selected from the group consisting of:

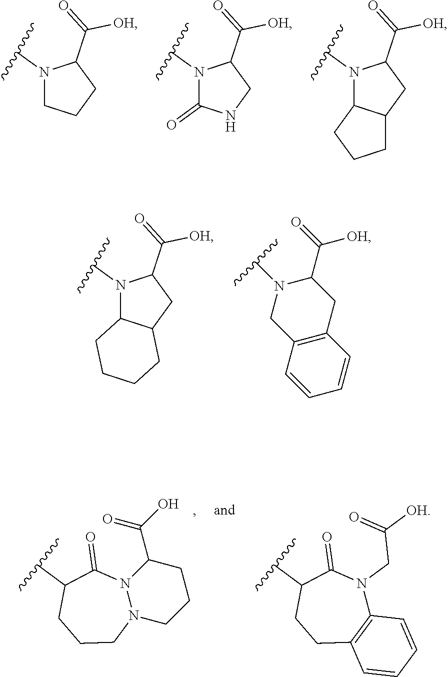

In one example, the angiotensin II signalling inhibitor according to Formula VI is:

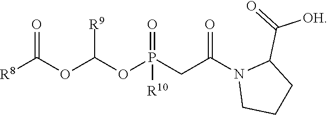

In one example, the angiotensin II signalling inhibitor according to Formula VI is

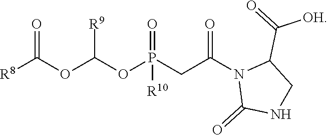

In one example, the angiotensin II signalling inhibitor according to Formula VI is

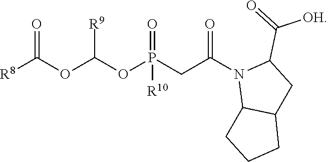

In one example, the angiotensin II signalling inhibitor according to Formula VI is

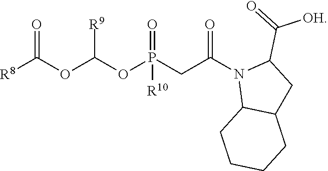

In one example, the angiotensin II signalling inhibitor according to Formula VI is

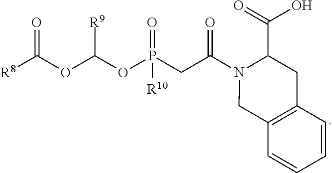

In one example, the angiotensin II signalling inhibitor according to Formula VI is

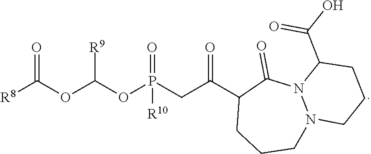

In one example, the angiotensin II signalling inhibitor according to Formula VI is

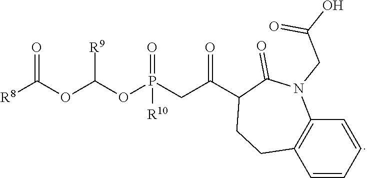

According to a compound of Formula VI, W may be optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, C₁₋₁₀alkylaryl, C₁₋₁₀alkylC₁₋₁₀ cyclyl, monocyclic or polycyclic carbocyclic, and monocyclic or polycyclic heterocyclic.

In one example, the angiotensin II signalling inhibitor according to Formula VI is

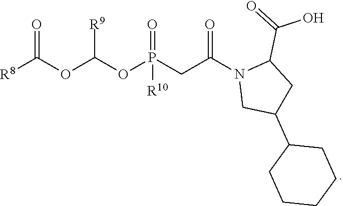

According to a compound of Formula VI, R⁸, R⁹ and R¹⁰ are each independently selected from the group consisting of hydrogen, halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, C₁₋₁₀alkylaryl, monocyclic or polycyclic carbocyclic, and monocyclic or polycyclic heterocyclic.

In one example, the angiotensin II signalling inhibitor according to Formula VI is

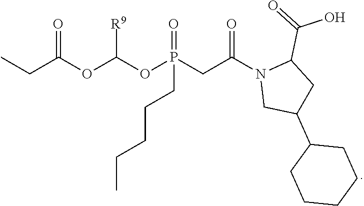

The C₁₋₁₀alkyl, C₂₋₁₀alkenyl, C₁₋₁₀alkylaryl, monocyclic or polycyclic carbocyclic, and monocyclic or polycyclic heterocyclic are each optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, and aminocarbonyl.

In one example, the angiotensin II signalling inhibitor according to Formula VI is

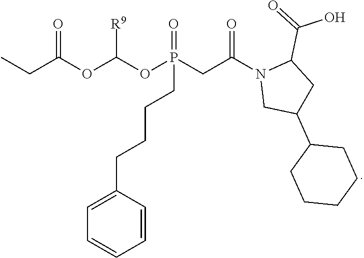

Further, according to a compound of Formula VI, the C₁₋₁₀alkyl, C₂₋₂₀alkenyl, and C₁₋₁₀alkylaryl, wherever each appears within Formula VI, may each be optionally interrupted with one or more heteroatoms independently selected from O, N and S.

In one example, the angiotensin II signalling inhibitor according to Formula VI is

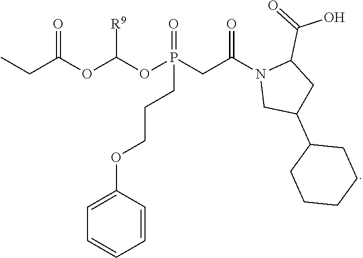

In one example, the angiotensin II signalling inhibitor according to Formula VI is Fosinopril.

In some embodiments, the angiotensin II signalling inhibitor has a structure according to Formula VII:

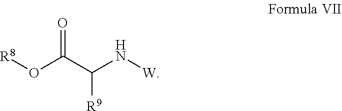

The above compounds of Formula VII may be further described as follows.

According to a compound of Formula VII, W is selected from the group consisting of:

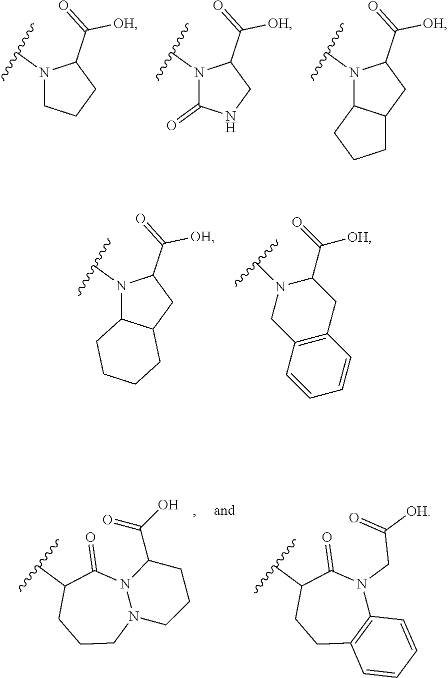

In one example, the angiotensin II signalling inhibitor according to Formula VII is

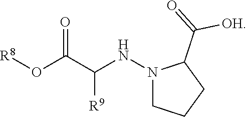

In one example, the angiotensin II signalling inhibitor according to Formula VII is

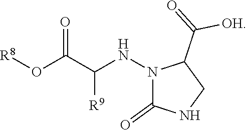

In one example, the angiotensin II signalling inhibitor according to Formula VII is

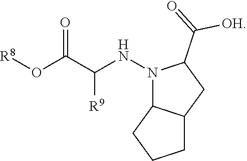

In one example, the angiotensin II signalling inhibitor according to Formula VII is

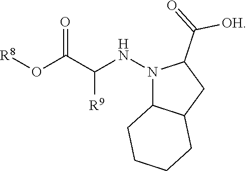

In one example, the angiotensin II signalling inhibitor according to Formula VII is

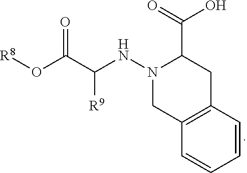

In one example, the angiotensin II signalling inhibitor according to Formula VII is

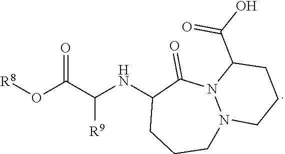

In one example, the angiotensin II signalling inhibitor according to Formula VII is

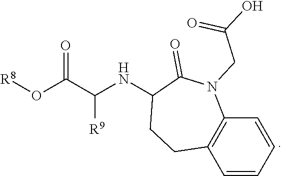

According to a compound of Formula VII, W may be optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, C₁₋₁₀alkylaryl, C₁₋₁₀alkylC₁₋₁₀ cyclyl, monocyclic or polycyclic carbocyclic, and monocyclic or polycyclic heterocyclic.

In one example, the angiotensin II signalling inhibitor according to Formula VII is

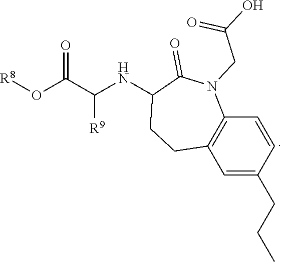

According to a compound of Formula VII, R⁸ and R⁹ are each independently selected from the group consisting of hydrogen, halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, C₁₋₁₀alkylaryl, monocyclic or polycyclic carbocyclic, and monocyclic or polycyclic heterocyclic.

In one example, the angiotensin II signalling inhibitor according to Formula VII is

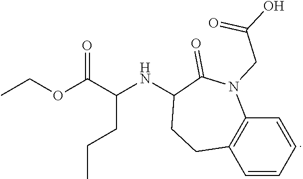 [SMILES: CCCC(NC1CCc2ccccc2N(CC(=O)O)C1=O)C(=O)OCC]

The C₁₋₁₀alkyl, C₂₋₁₀alkenyl, C₁₋₁₀alkylaryl, monocyclic or polycyclic carbocyclic, and monocyclic or polycyclic heterocyclic are each optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, and aminocarbonyl.

In one example, the angiotensin II signalling inhibitor according to Formula VII is

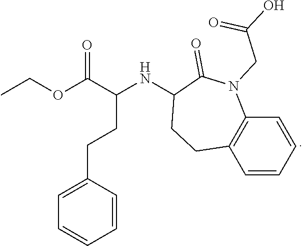 [SMILES: CCOC(=O)C(CCc1ccccc1)NC1CCc2ccccc2N(CC(=O)O)C1=O]

Further, according to a compound of Formula VII, the C₁₋₁₀alkyl, C₂₋₂₀alkenyl, and C₁₋₁₀alkylaryl, wherever each appears within Formula VII, may each be optionally interrupted with one or more heteroatoms independently selected from O, N and S.

In one example, the angiotensin II signalling inhibitor according to Formula VII is

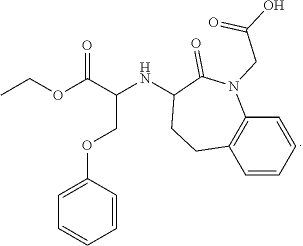 [SMILES: CCOC(=O)C(COc1ccccc1)NC1CCc2ccccc2N(CC(=O)O)C1=O]

In one example, the angiotensin II signalling inhibitor according to Formula VII is Cilazapril. In one example, the angiotensin II signalling inhibitor according to Formula VII is Benazepril.

In some embodiments, the angiotensin II signalling inhibitor has a structure according to Formula VIII:

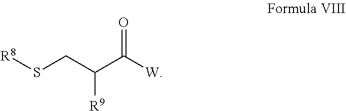

The above compounds of Formula VIII may be further described as follows.

According to a compound of Formula VIII, W is selected from the group consisting of:

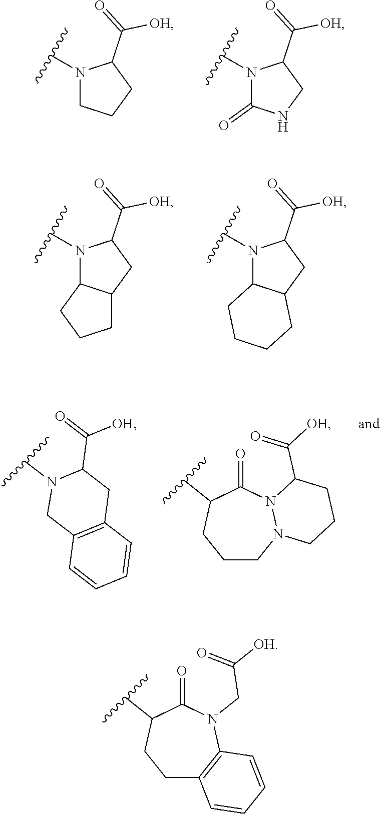

In one example, the angiotensin II signalling inhibitor according to Formula VIII is:

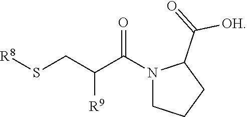

In one example, the angiotensin II signalling inhibitor according to Formula VIII is

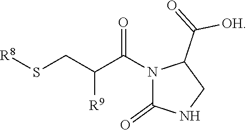

In one example, the angiotensin II signalling inhibitor according to Formula VIII is

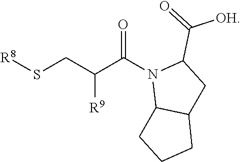

In one example, the angiotensin II signalling inhibitor according to Formula VIII is

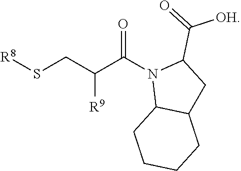

In one example, the angiotensin II signalling inhibitor according to Formula VIII is

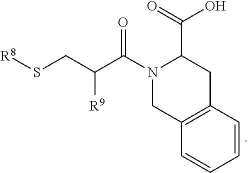

In one example, the angiotensin II signalling inhibitor according to Formula VIII is

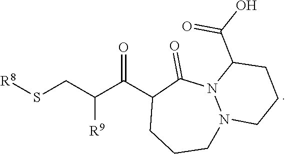

In one example, the angiotensin II signalling inhibitor according to Formula VIII is

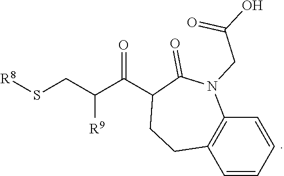

According to a compound of Formula VIII, W may be optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, C₁₋₁₀alkylaryl, C₁₋₁₀alkylC₁₋₁₀ cyclyl, monocyclic or polycyclic carbocyclic, and monocyclic or polycyclic heterocyclic.

In one example, the angiotensin II signalling inhibitor according to Formula VIII is

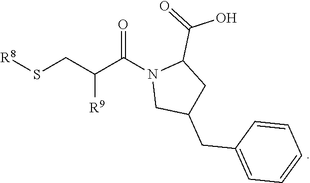

According to a compound of Formula VIII, R⁸ and R⁹ are each independently selected from the group consisting of hydrogen, halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, C₁₋₁₀alkylaryl, monocyclic or polycyclic carbocyclic, and monocyclic or polycyclic heterocyclic.

In one example, the angiotensin II signalling inhibitor according to Formula VIII is

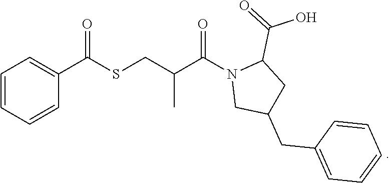 [SMILES: CC(CSC(=O)c1ccccc1)C(=O)N1CC(Cc2ccccc2)CC1C(=O)O]

The C₁₋₁₀alkyl, C₂₋₁₀alkenyl, C₁₋₁₀alkylaryl, monocyclic or polycyclic carbocyclic, and monocyclic or polycyclic heterocyclic are each optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, and aminocarbonyl.

In one example, the angiotensin II signalling inhibitor according to Formula VIII is

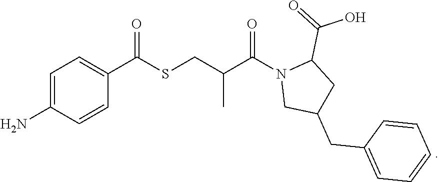 [SMILES: CC(CSC(=O)c1ccc(N)cc1)C(=O)N1CC(Cc2ccccc2)CC1C(=O)O]

Further, according to a compound of Formula VIII, the C₁₋₁₀alkyl, C₂₋₂₀alkenyl, and C₁₋₁₀alkylaryl, wherever each appears within Formula VIII, may each be optionally interrupted with one or more heteroatoms independently selected from O, N and S.

In one example, the angiotensin II signalling inhibitor according to Formula VIII is

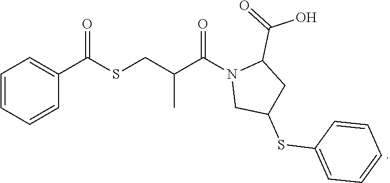 [SMILES: CC(CSC(=O)c1ccccc1)C(=O)N1CC(Sc2ccccc2)CC1C(=O)O]

In one example, the angiotensin II signalling inhibitor according to Formula VIII is Zofenopril.

It will be appreciated that any of the optional heteroatoms or substituents referred to above in Formula V, VI, VII, or VIII, with reference to “one or more”, unless otherwise stated, may be any integer such as 1, 2, 3, 4, 5, 6, etc., or for example a range of 1 to 6 substituents, 1 to 3 substituents, or 1 to 2 substituents.

A number of small molecule angiotensin-converting enzyme (ACE) inhibitors are known to exist, and there are examples where angiotensin-converting enzyme (ACE) inhibitors have reached the market. Examples include, but are not limited to, Captopril (trade name Capoten), Quinapril (Accupril), Imidapril (Tanatril), Zofenopril (Bifril/Zofenil), Enalapril (Vasotec/Renitec/Berlipril/Enap/Enalapril/Profarma), Cilazapril (Inhibace), Ramipril (Altace/Prilace/Ramace/Ramiwin/Triatec/Tritace), Benazepril (Lotensin), Perindopril (Coversyl/Aceon/Perindo), Trandolapril (Mavik/Odrik/Gopten), Lisinopril (Listril/Lopril/Novatec/Prinivil/Zestril/Lisidigal), Fosinopril (Fositen/Monopril), and Moexipril (Univasc) (Table 2). In one example, the angiotensin II signalling inhibitor is Captopril. In one example, the angiotensin II signalling inhibitor is Quinapril. In one example, the angiotensin II signalling inhibitor is Imidapril. In one example, the angiotensin II signalling inhibitor is Zofenopril. In one example, the angiotensin II signalling inhibitor is Enalapril. In one example, the angiotensin II signalling inhibitor is Cilazapril. In one example, the angiotensin II signalling inhibitor is Ramipril. In one example, the angiotensin II signalling inhibitor is Benazepril. In one example, the angiotensin II signalling inhibitor is Perindopril. In one example, the angiotensin II signalling inhibitor is Trandolapril. In one example, the angiotensin II signalling inhibitor is Lisinopril. In one example, the angiotensin II signalling inhibitor is Fosinopril. In one example, the angiotensin II signalling inhibitor is Moexipril. In one example, the angiotensin-converting enzyme (ACE) inhibitor is Captopril.

TABLE 2
Structures of Known Angiotensin-Converting Enzyme (ACE)
Inhibitors

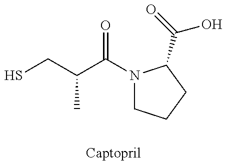

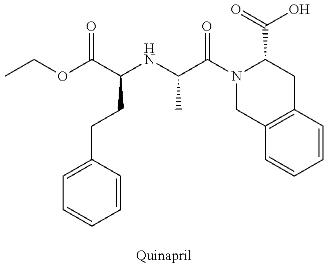 [SMILES: CCOC(=O)[C@H](CCc1ccccc1)N[C@@H](C)C(=O)N1Cc2ccccc2C[C@H]1C(=O)O]

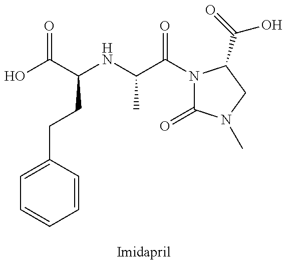 [SMILES: C[C@H](N[C@@H](CCc1ccccc1)C(=O)O)C(=O)N1C(=O)N(C)C[C@H]1C(=O)O]

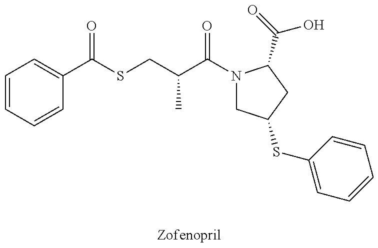 [SMILES: C[C@H](CSC(=O)c1ccccc1)C(=O)N1C[C@@H](Sc2ccccc2)C[C@H]1C(=O)O]

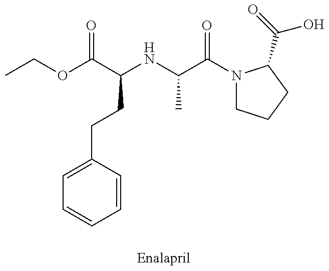 [SMILES: CCOC(=O)[C@H](CCc1ccccc1)N[C@@H](C)C(=O)N1CCC[C@H]1C(=O)O]

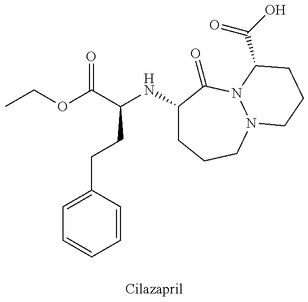 [SMILES: CCOC(=O)[C@H](CCc1ccccc1)N[C@H]1CCCN2CCC[C@@H](C(=O)O)N2C1=O]

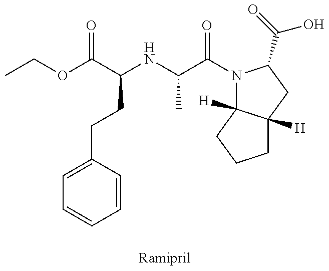 [SMILES: CCOC(=O)[C@H](CCc1ccccc1)N[C@@H](C)C(=O)N1[C@H](C(=O)O)C[C@@H]2CCC[C@@H]21]

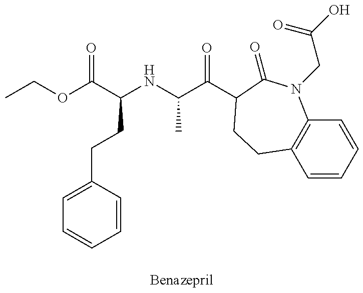 [SMILES: CCOC(=O)[C@H](CCc1ccccc1)N[C@@H](C)C(=O)C1CCc2ccccc2N(CC(=O)O)C1=O]

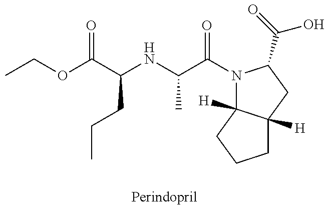 [SMILES: CCC[C@H](N[C@@H](C)C(=O)N1[C@H](C(=O)O)C[C@@H]2CCC[C@@H]21)C(=O)OCC]

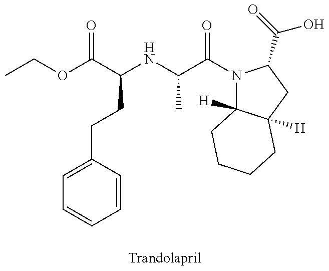 [SMILES: CCOC(=O)[C@H](CCc1ccccc1)N[C@@H](C)C(=O)N1[C@H](C(=O)O)C[C@H]2CCCC[C@@H]21]

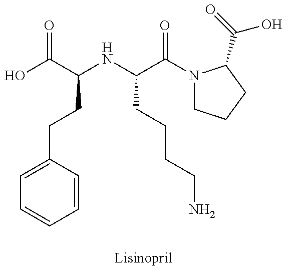 [SMILES: NCCCC[C@H](N[C@@H](CCc1ccccc1)C(=O)O)C(=O)N1CCC[C@H]1C(=O)O]

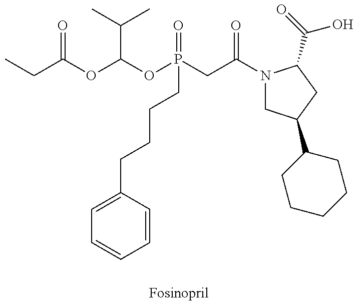 [SMILES: CCC(=O)OC(OP(=O)(CCCCc1ccccc1)CC(=O)N1C[C@H](C2CCCCC2)C[C@H]1C(=O)O)C(C)C]

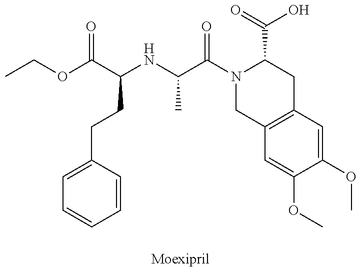 [SMILES: CCOC(=O)[C@H](CCc1ccccc1)N[C@@H](C)C(=O)N1Cc2cc(OC)c(OC)cc2C[C@H]1C(=O)O]

### Renin Inhibitors

In some embodiments, the angiotensin II signalling inhibitor is a renin inhibitor. As used herein, the term “renin inhibitor” refers to a molecule that inhibits the biological activity of renin. Renin, also known as angiotensinogenase, is a protein and enzyme secreted by the kidneys that is responsible for hydrolysing angiotensinogen to angiotensin I. In one example, the angiotensin II signalling inhibitor is a renin inhibitor.

In some embodiments, the angiotensin II signalling inhibitor has a structure according to Formula IX:

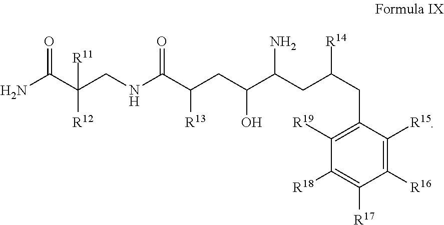

The above compounds of Formula IX may be further described as follows.

According to a compound of Formula IX, R¹¹, R¹², R¹³, R¹⁴, R¹⁵, R¹⁶, R¹⁷, R¹⁸, and R¹⁹ are each independently selected from the group consisting of hydrogen, halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, C₁₋₁₀ alkylaryl, monocyclic or polycyclic carbocyclic, and monocyclic or polycyclic heterocyclic.

In one example, the angiotensin II signalling inhibitor according to Formula IX is

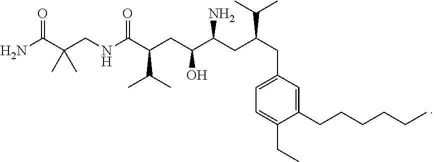 [SMILES: CCCCCCc1cc(C[C@@H](C[C@H](N)[C@@H](O)C[C@H](C(=O)NCC(C)(C)C(N)=O)C(C)C)C(C)C)ccc1CC]

Further, according to a compound of Formula IX, the C₁₋₁₀alkyl, C₂₋₂₀alkenyl, and C₁₋₁₀alkylaryl, wherever each appears within Formula IX, may each be optionally interrupted with one or more heteroatoms independently selected from O, N and S.

In one example, the angiotensin II signalling inhibitor according to Formula IX is

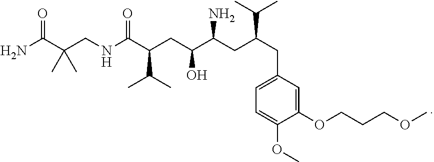 [SMILES: COCCCOc1cc(C[C@@H](C[C@H](N)[C@@H](O)C[C@H](C(=O)NCC(C)(C)C(N)=O)C(C)C)C(C)C)ccc1OC]

In one example, the angiotensin II signalling inhibitor according to Formula IX is Aliskiren.

It will be appreciated that any of the optional heteroatoms or substituents referred to above in Formula IX, with reference to “one or more”, unless otherwise stated, may be any integer such as 1, 2, 3, 4, 5, 6, etc., or for example a range of 1 to 6 substituents, 1 to 3 substituents, or 1 to 2 substituents.

A small molecule renin inhibitor is known to exist and to have reached the market. This example is Aliskiren (trade name Tekturna/Rasilez) (Table 3). In one example, the angiotensin II signalling inhibitor is Aliskiren. In one example, the renin inhibitor is Aliskiren.

TABLE 3
Structure of Known Renin Inhibitor

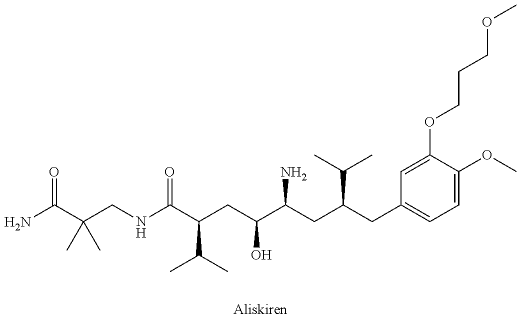 [SMILES: COCCCOc1cc(C[C@@H](C[C@H](N)[C@@H](O)C[C@H](C(=O)NCC(C)(C)C(N)=O)C(C)C)C(C)C)ccc1OC]

### Method of Treatment

It has been surprisingly found that inhibiting the angiotensin II signalling pathway provides a method of treating or preventing a Mononegavirales viral infection. Specifically, it has been surprisingly found that inhibiting the angiotensin II signalling pathway blocks viral RNA replication in a cell infected with a Mononegavirales virus. In some embodiments, there is provided a method of treating or preventing a Mononegavirales viral infection in a subject, the method comprising administering an effective amount of an angiotensin II signalling inhibitor wherein the angiotensin II signalling inhibitor blocks viral RNA replication. In one example, there is provided a method of treating or preventing a Mononegavirales viral infection in a subject, the method comprising administering an effective amount of an angiotensin II signalling inhibitor.

### Pre-Existing or Other Health Conditions

In some embodiments, the subject may or may not have pre-existing health conditions other than the viral infection. The angiotensin II signalling pathway is associated with the regulation of blood pressure and cardiac events, and consequently, inhibitors of the angiotensin II signalling pathway are administered in the treatment of such diseases, including, for example, endothelial dysfunction, hypertension (e.g., high blood pressure), diabetic nephropathy, and congestive heart failure. Such pre-existing health conditions may or may not be associated with a viral infection. That is, a subject may contract a viral infection independent of suffering from a pre-existing health condition, or the subject may be suffering from a pre-existing health condition that is associated with a viral infection. In one example, the subject having the viral infection is not suffering from a pre-existing condition. In one example, the subject having the viral infection is suffering from a pre-existing condition.

The pre-existing health condition may or may not have been diagnosed by a medical practitioner. That is, the subject having the viral infection may be suffering from an undiagnosed pre-existing health condition. In one example, the subject having the viral infection is suffering from a diagnosed pre-existing condition. In one example, the subject having the viral infection is suffering from an undiagnosed pre-existing condition.

It is possible that a subject is being administered with an angiotensin II signalling inhibitor (e.g., angiotensin II receptor antagonist, angiotensin-converting enzyme (ACE) inhibitor, renin inhibitor) for the treatment of a condition associated with the angiotensin II signalling pathway (e.g., endothelial dysfunction, hypertension, diabetic nephropathy, and congestive heart failure) at the time of contracting a viral infection. In one example, the subject is being treated for a condition associated with the angiotensin II signalling pathway. Otherwise, it is possible that a subject is not suffering from a condition associated with the angiotensin II signalling pathway (e.g., endothelial dysfunction, hypertension, diabetic nephropathy, and congestive heart failure) at the time of contracting a viral infection. In one example, the subject is not being treated for a condition associated with the angiotensin II signalling pathway (e.g., endothelial dysfunction, hypertension, diabetic nephropathy, and congestive heart failure). In one example, the subject is not being treated for endothelial dysfunction. In one example, the subject is not being treated for hypertension. In one example, the subject is not being treated for diabetic nephropathy. In one example, the subject is not being treated for congestive heart failure.

### Administration

In some embodiments, the angitotensin II signalling inhibitor is administered to the subject by various routes, e.g., oral, topical, subcutaneous, transdermal, intramuscular, intravenous, or intraperitoneal. Several angiotensin II signalling inhibitors (e.g., angiotensin II receptor antagonists, angiotensin-converting enzyme (ACE) inhibitors, renin inhibitors) are marketed for oral delivery. That is, the majority of angiotensin II receptor antagonists, angiotensin-converting enzyme (ACE) inhibitors, and renin inhibitors are marketed as being orally administrable. It is therefore appreciated that such known drugs exhibit the appropriate properties, i.e., pharmacokinetic and physicochemical properties, to be biopharmaceutically active upon oral administration. In one example, the angiotensin II signalling inhibitor is administered to the subject orally. In one example, the angiotensin II signalling inhibitor is administered to the subject intravenously.

Similarly, such known drugs are prescribed in particular dosage amounts. For example, the angiotensin II receptor antagonist, Telmisartan, is prescribed for the treatment of hypertension and is available as 20 mg, 40 mg, and 80 mg oral dosage amounts. In the treatment of a viral infection, the angiotensin II signalling inhibitor may be administered in a dosage amount prescribed for the treatment of its previously known use, e.g., Telmisartan in the treatment of hypertension, or may be administered in a dosage amount that differs from the amount prescribed for the treatment of its previously known use. That is, the dosage amount of the angiotensin II signalling inhibitor required to be administered for the treatment of a viral infection is independent of the dosage amount prescribed for the treatment of its previously known use. In one example, the amount of the angiotensin II signalling inhibitor required to be administered for the treatment of a viral infection is independent of the dosage amount prescribed for the treatment of its previously known use.

Similarly, such known drugs are prescribed in particular dosage regimes. For example, the angiotensin II receptor antagonist, Telmisartan, is prescribed for the treatment of hypertension initially as a 40 mg dose one daily, and may be increased to a one daily dose of 80 mg. In the treatment of a viral infection, the angiotensin II signaling inhibitor may be administered according to a dosage regime prescribed for the treatment of its previously known use, e.g., Telmisartan in the treatment of hypertension, or may be administered according to a different dosage regime than that prescribed for the treatment of its previously known use. That is, the dosage regime of the angiotensin II signalling inhibitor required to be administered for the treatment of a viral infection is independent of the dosage regime prescribed for the treatment of its previously known use.

In one example, the dosage regime of the angiotensin II signalling inhibitor administered for the treatment of a viral infection is independent of the dosage regime prescribed for the treatment of its previously known use.

### Combination Therapies

In some embodiments, the angiotensin II signalling inhibitor is administered to the subject as a single therapy for the treatment or prevention of a viral infection. That is, a single angiotensin II signalling inhibitor, such as Telmisartan, is administered to the subject for the treatment or prevention of a viral infection. In one example, the angiotensin II signalling inhibitor is administered to the subject as a single therapy for the treatment or prevention of a viral infection.

In some embodiments, the angiotensin II signalling inhibitor is administered to the subject as a combination therapy for the treatment or prevention of a viral infection. That is, a combination of angiotensin II signalling inhibitors, such as Telmisartan and Candesartan, is administered to the subject for the treatment or prevention of a viral infection. In one example, a combination of angiotensin II signalling inhibitors is administered to the subject for the treatment or prevention of a viral infection.

The combination of angiotensin II signalling inhibitors includes two, three, four, five, six, seven, eight, nine, ten, etc. different angiotensin II signalling inhibitors. In one example, a combination of two angiotensin II signalling inhibitors is administered to a subject for the treatment of a viral infection. In one example, a combination of three angiotensin II signalling inhibitors is administered to a subject for the treatment of a viral infection.

The combination of angiotensin II signalling inhibitors may include inhibitors from the same class (i.e., two, three, four, five, six, seven, eight, nine, ten, etc., angiotensin II receptor antagonists). Alternatively, the combination of angiotensin II signalling inhibitors may include inhibitors from one or more different classes. In one example, a combination of one or more angiotensin II receptor antagonists and one or more angiotensin-converting enzyme (ACE) inhibitors is prescribed. In one example, a combination of one or more angiotensin II receptor antagonists and one or more renin inhibitors is prescribed. In one example, a combination of one or more angiotensin-converting enzyme (ACE) inhibitors and one or more renin inhibitors is prescribed. It will be understood that all possible combinations of angiotensin II signalling inhibitors may be prescribed in the treatment of a viral infection. For example, a combination of two angiotensin II receptor antagonists and one angiotensin-converting enzyme (ACE) inhibitor is prescribed. In one example, a combination of Telmisartan and Candesartan is administered to the subject for the treatment or prevention of a viral infection.

In some embodiments, the angiotensin II signalling inhibitor, or combination thereof, is administered to the subject as a therapy for the treatment or prevention of a viral infection in combination with another antiviral compound. Such other antiviral compounds include, for example, Abacavir, Aciclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ihacitabine, Imunovir, Idoxuridine, Imiquimod, Indinavir, Inosine, Interferon type III, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Novir, Oseltamivir, Peginterferon alfa-2a, Penciclovir, Peramivir, Pleconaril, Podophyllotoxin, Raltegravir, Ribavirin, Rimantadine, Ritonavir, Pyramidine, Saquinavir, Sofosbuvir, Stavudine, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, and Zidovudine. A combination of any two or more other antiviral compounds may be administered with the angiotensin II signalling inhibitor. It will be understood that all possible combinations of angiotensin II signalling inhibitors in combination with all combinations of another antiviral compound may be prescribed in the treatment of a viral infection. In one example, a combination of an angiotensin II signalling inhibitor and another antiviral is administered to the subject for the treatment or prevention of a viral infection. In one example, a combination of Telmisartan and another antiviral compound is administered to the subject for the treatment or prevention of a viral infection. In one example, a combination of Telmisartan, Candesartan and another antiviral compound is administered to the subject for the treatment or prevention of a viral infection.

### Formulations

The person skilled in the art will appreciate that the angiotensin II signalling inhibitors may be appropriately formulated into a pharmaceutical composition for administration to the subject. The pharmaceutical compositions may be suitable for use in a variety of drug delivery systems. Suitable formulations for use in the present disclosure may be found in Remington's Pharmaceutical Sciences, Mack Publishing Company, Philadelphia, Pa., 17th ed. (1985). For a brief review of methods for drug delivery, see Langer (1990).

For preparing pharmaceutical compositions containing angiotensin II signalling inhibitors, inert and pharmaceutically acceptable carriers are used. As used herein, the term “pharmaceutically acceptable carrier” includes any and all solids or solvents (such as phosphate buffered saline buffers, water, saline) dispersion media, coatings, antibacterial and antifungal agents, isotonic and absorption delaying agents, and the like, compatible with pharmaceutical administration. The pharmaceutically acceptable carriers must be ‘acceptable’ in the sense of being compatible with the other ingredients of the composition and not deleterious to the recipient thereof. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material. In powders, the carrier is generally a finely divided solid that is in a mixture with the finely divided active component. In tablets, the active ingredient (an angiotensin II signalling inhibitor) is mixed with the carrier having the necessary binding properties in suitable proportions and compacted in the shape and size desired.

The amount of pharmaceutically acceptable carrier will depend upon the level of the compound and any other optional ingredients that a person skilled in the art would classify as distinct from the carrier (e.g., other active agents). The formulations of the present invention may comprise, for example, from about 5% to 99.99%, or 25% to about 99.9% or from 30% to 90% by weight of the composition, of a pharmaceutically acceptable carrier. The pharmaceutically acceptable carrier can, in the absence of other adjuncts, form the balance of the composition.

The amount of pharmaceutically acceptable carrier will depend upon the level of the compound and any other optional ingredients that a person skilled in the art would classify as distinct from the carrier (e.g., other active agents). The formulations of the present invention may comprise, for example, from about 5% to 99.99%, or 25% to about 99.9% or from 30% to 90% by weight of the composition, of a pharmaceutically acceptable carrier. The pharmaceutically acceptable carrier can, in the absence of other adjuncts, form the balance of the composition.

Optionally, the pharmaceutical composition of the present disclosure further comprises other additional components, for example therapeutic and/or prophylactic ingredients. The invention thus relates in a further aspect to pharmaceutical composition comprising the compound of the present invention, one or more pharmaceutically acceptable carriers together with one or more other active agents. Generally, the amount of other active agent present in the pharmaceutical composition is sufficient to provide an additional benefit either alone or in combination with the other ingredients in the composition.

It will be understood by the person skilled in the art that these optional components may be categorized by their therapeutic or aesthetic benefit or their postulated mode of action. However, it is also understood that these optional components may, in some instances, provide more than one therapeutic or aesthetic benefit or operate via more than one mode of action. Therefore, classifications herein are made for the sake of convenience and are not intended to limit the component to that particular application or applications listed. Also, when applicable, the pharmaceutically-acceptable salts of the components are useful herein.

When other active agents are present in the pharmaceutical formulation of the present invention, the dose of the compound may either be the same as or differ from that employed when the other additional components are not present. Appropriate doses will be readily appreciated by those skilled in the art.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets may contain between about 5% to about 70% by weight of the active ingredient of an angiotensin II signalling inhibitor. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound of an angiotensin II signalling inhibitor with encapsulating material as a carrier providing a capsule in which the inhibitor (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., an angiotensin II signalling inhibitor) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., an angiotensin II signalling inhibitor) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, for example from 5 to 9, or from 7 to 8.

Single or multiple administrations of the pharmaceutical compositions can be carried out with dose levels and pattern being selected by the treating practitioner. In any event, the pharmaceutical formulations should provide a quantity of an angiotensin II signalling inhibitor sufficient to effectively treat or prevent a viral infection in the patient.

When used for pharmaceutical purposes, the angiotensin II signalling inhibitor inhibitor may be generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. (1966).

The compositions can additionally include a stabilizer, enhancer or other pharmaceutically acceptable carriers or vehicles. A pharmaceutically acceptable carrier can contain a physiologically acceptable compound that acts, for example, to stabilize the compounds. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

The formulations containing an angiotensin II signalling inhibitor may be delivered to any tissue or organ using any delivery method known to the ordinarily skilled artisan. They may be formulated for subcutaneous, intramuscular, intravenous, intraperitoneal, or intratumor injection, or for oral ingestion or for topical application.

Effective dosage of the formulations will vary depending on many different factors, including means of administration, target site, physiological state of the patient, and other medicines administered. Thus, treatment dosages will need to be titrated to optimize safety and efficacy. In determining the effective amount of a compound to be administered, the physician should evaluate the particular compound being used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. Doses may generally range between about 0.01 and about 100 μg per kilogram of body weight, for example between about 0.1 and about 50 μg per kg of body weight.

In some embodiments, the pharmaceutical composition comprises a therapeutically effective amount of an angiotensin II signalling inhibitor. The content of the angiotensin II signalling inhibitor in the pharmaceutical composition is, for example, from about 0.1% to about 100% w/w of the pharmaceutical composition. In one example, the pharmaceutical composition comprises a therapeutically effective amount of an angiotensin II receptor antagonist. In one example, the pharmaceutical composition comprises a therapeutically effective amount of an angiotensin-converting enzyme (ACE) inhibitor. In one example, the pharmaceutical composition comprises a therapeutically effective amount of a renin inhibitor. In one example, the pharmaceutical composition comprises a therapeutically effective amount of Telmisartan. In one example, the pharmaceutical composition comprises a therapeutically effective amount of Candesartan. In one example, the pharmaceutical composition comprises a therapeutically effective amount of an angiotensin II receptor antagonist and another antiviral compound.

The present disclosure provides pharmaceutical formulations or compositions, both for veterinary and for human medical use, which comprise one or more angiotensin II signalling inhibitors, which may or may not be in combination with one or more other antiviral compounds, or any embodiments thereof as described herein or any pharmaceutically acceptable salts thereof, with one or more pharmaceutically acceptable carriers and/or excipients, and optionally any other therapeutic ingredients, stabilisers, or the like.

The carrier(s) or excipients must be pharmaceutically acceptable in the sense of being compatible with the other ingredients, such as sugars, hydroxyethylstarch (HES), dextrates (e.g., cyclodextrins, such as 2-hydroxypropyl-β-cyclodextrin and sulfobutylether-β-cyclodextrin), polyethylene glycols, and pectin. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

The present disclosure will now be described further with reference to the following examples, which are illustrative only and non-limiting. The examples refer to the figures.

### EXAMPLES

### Example 1—Antiviral Properties of Angiotensin II Signalling Inhibitors

### Materials and Methods

Cells:

HeLa cells (ATCC CCL-2) were maintained in growth media (EMEM supplemented with 10% (v/v) foetal calf serum (FCS), 10 mM HEPES, 2 mM L-glutamine, 100 U/mL penicillin and 100 μg/mL streptomycin. African green monkey kidney epithelial Vero cells (ATCC CRL-81) were maintained in DMEM supplemented with 10% (v/v) FCS, 100 U/mL penicillin and 100 μg/mL streptomycin. All cells were incubated at 37° C. under a 5% CO₂/95% air atmosphere.

Viruses:

All virology work was conducted at the CSIRO Australian Animal Health Laboratory. HeV (Hendra virus/horse/1994/Hendra), NiV (Nipah virus/Malaysia/human/99) and RSV (strain A2) were passaged in Vero cells. Influenza A/WSN/33 (H1N1) (kind gift, Professor Lorena Brown, University of Melbourne) was passaged in the allantoic fluid of 10-day embryonated specific pathogen-free chicken eggs (Australian SPF Services, Cadello, VIC, Australia). All viruses were aliquoted and stored at −80° C. for inoculations.

Chemical Compound Screen:

A boutique chemical compound library screen was performed in collaboration with the Walter and Eliza Hall Institute High Throughput Chemical Screening Facility (Bundoora, Australia). The library consisted of four sub-libraries: epigenetics (77 compounds), kinase inhibitors (210 compounds), known drugs (3707 compounds) and targeted agents (73 compounds). The known drugs library is a compilation of 3 commercially available libraries: Tocris, Prestwick and LOPAC. Compounds were obtained from the Queensland Compound Library (Queensland, Australia) and stored in neat dimethyl sulfoxide (DMSO) in 384 well black walled, clear-bottomed tissue culture plates. Positive controls were included on every plate. E64D (Sigma # E8640) and calpain inhibitor II (Sigma # A6060) were assayed at 2 μM and 10 μM. Both molecules inhibit the cathepsin L protease required for the cleavage and activation of HeV fusion protein, as described in Pager and Dutch (2005).

HeLa cells (4000 cells/well) were added to plates containing compounds using a BioTek 406 liquid handling robot (BioTek, Winooski, Vt.). After 1 h, cells were infected with HeV (multiplicity of infection 0.1 for 24 h) at bio-safety level (BSL-4). After this time, cells were fixed with 4% paraformaldehyde for 2 h, removed from BSL-4 and immustained for HeV antigen detection. Cells were permeabilised with 0.1% Triton X-100 (Sigma) in PBS for 10 min and non-specific binding was blocked with 0.5% bovine serum albumin (BSA) (Sigma) in PBS for 30 min. A rabbit anti-HeV nucleoprotein (HeV-N) antibody (AAHL) was diluted 1:1000 in PBS-BSA and incubated on cells for 1 h followed by 3×5 min PBS washes and a 1 h incubation with anti-rabbit Alexa Fluor 488 antibody (A11008, Life Technologies) at 1:200 in PBS-BSA. Cells were washed 3×5 min with PBS, then stained with the nuclear stain 4′,6-diamidino-2-phenylindole dihydrochloride (DAPI) (Invitrogen, Carlsbad, Calif.; 1 μg/ml) for 20 min in PBS. Cells were imaged using a CellInsight Personal Cell Imager (Thermo Scientific) using a 20× objective, with all fields per well imaged, excluding edges. The percentage of infected cells, the mean fluorescence per infected cell, and the number of cell nuclei per well were quantitated using the Target Activation bioapplication of the Cellomics Scan software (iDev workflow) (Thermo Fisher, Waltham, Mass.).

Tissue Culture Infective Dose (TCID₅₀) Analysis:

TCID₅₀ assays were performed as described in Deffrasnes et al. (2016) and Foo et al. (2016). Infectious virus titre was then calculated as described in Reed and Muench (1938).

Quantitative Real-Time PCR:

Quantitative RT-PCR for HeV RNA was performed as described in Deffrasnes et al. (2016) and Foo et al. (2016).

Cell-Cell Fusion Assay:

HeV-F and -G mediated fusion assays were performed as described in Deffrasnes et al. (2016) and Foo et al. (2016).

Cell Viability:

Cells transfected in a 96-well plate with 40 nM siRNAs were fixed 72 h posttransfection with 4% paraformaldehyde. Cell nuclei were then stained using 4′,6-diamidino-2-phenylindole dihydrochloride (DAPI) nuclei stain (Invitrogen), and the number of viable nuclei per treatment group was quantitated using the CellInsight Personal Cell Imager (Thermo Scientific, Waltham, Mass.).

Statistics:

The difference between two groups was statistically analysed by a two-tailed Student's t-test. A p-value of <0.05 was considered significant. **p<0.01, *p<0.05, N.S. not significant. All data points are the average of triplicates, with error bars representing standard deviation. All data is representative of at least 2 separate experiments.

### Results

Screening of Drug Library for Inhibitors of HeV Infection:

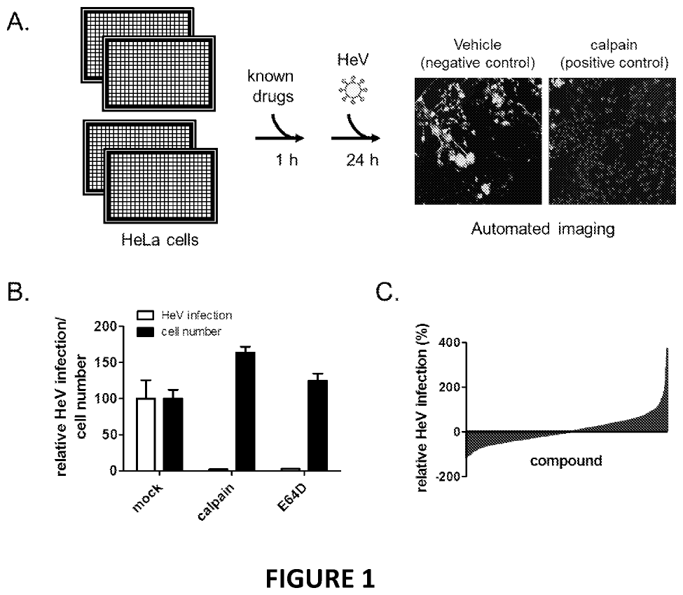

A library of 4,148 chemical compounds was screened for inhibition of HeV infection in HeLa cells. Compounds were assayed at final concentrations of 1 μM and 10 μM, with a single well for each treatment group. Cells were incubated with compounds for 1 h, followed by infection with wild-type HeV for 24 h in a BSL-4 laboratory. Following infection, cells were fixed with 4% paraformaldehyde for 2 h, and removed to a BSL-3 laboratory, where cells were stained with antibodies to detect HeV antigen, and DAPI nuclear dye to innumerate cell numbers. A workflow for this process is shown in FIG. 1A.

Negative and positive controls were used to evaluate HeV inhibition and assay readout robustness on a per-plate basis. As a positive control for reducing HeV infection, cells were separately incubated with two chemical compound inhibitors of cathepsin L, E64D and calpain inhibitor II, at 10 μM. As a negative control, cells were incubated with the appropriate level of compound diluents (DMSO diluted in phosphate-buffered saline (PBS)). As the multiplicity of infection of HeV used in the screen resulted in some cell death (FIG. 1B), compounds that inhibited virus resulted in an increase in cell numbers per well and decrease in virus antigen levels per well, compared to negative control wells (FIG. 1B). These results indicated that calpain inhibitor II was more effective than E64D at inhibiting virus-induced cell death and viral protein synthesis.

A normalized view of cell number/well resulting from the screen is shown in FIG. 1C. Percentage cell number and percentage virus antigen levels are normalised to DMSO (negative control) and calpain inhibitor II 10 μM (positive control) as lower (0%) and upper (100%) signal respectively.

Telmisartan Inhibits Infection by HeV and Other Negative Strand RNA Viruses:

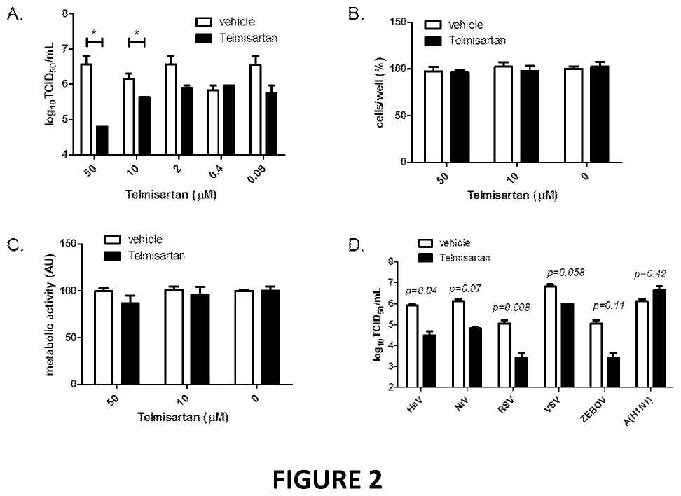

The screening identified Telmisartan as a novel antiviral therapy against HeV and related viruses. The impact of Telmisartan on HeV infection was intriguing given that there have been no reports of its antiviral properties described previously. As Telmisartan is an orally active compound that with known toxicity and safety profiles for human use, its impact on virus infection was investigated in greater detail. Firstly, the impact of Telmisartan on the production of infectious wild-type HeV was assessed. Telmisartan inhibited HeV infection in a dose-dependent manner (FIG. 2A). A significant reduction (˜95%) in HeV titers was observed in supernatant collected 24 h after infection with cells treated with the highest level of Telmisartan (50 μM).

The impact of Telmisartan on cell health was next assessed. Treating HeLa cells with Telmisartan at concentrations resulting in antiviral activity showed a mild impact on cell numbers (FIG. 2B). An Alamar blue assay showed no significant change in metabolic activity in cells treated with Telmisartan for 26 h compared to cells treated with equivalent levels of vehicle or media (FIG. 2C).

Whether other paramyxovirus infections could be blocked by Telmisartan was next investigated. Members of the family Paramyxoviridae are divided into two subfamilies (Paramyxovirinae and Pneumovirinae) where HeV and NiV belong to the genus Henipavirus in the subfamily Paramyxovirinae. Viruses belonging to different genera in the same subfamily were tested: Nipah virus, and a virus belonging to the subfamily Pneumoviridae, RSV (genus Pneumovirus). In addition, infection by three other viruses from the Mononegavirales: influenza A virus (A/WSN/33) (family Orthomyxoviridae), vesicular stomatitis virus (VSV, family Rhabdoviridae) and Zaire Ebolavirus (family Filoviridae) was tested. A significant reduction in virus titres for HeV, NiV, RSV, VSV but not for A/WSN/33 in cells treated with Telmisartan (FIG. 2D) was observed.

Telmisartan Inhibits the Early Stages of HeV Infection Post-Entry:

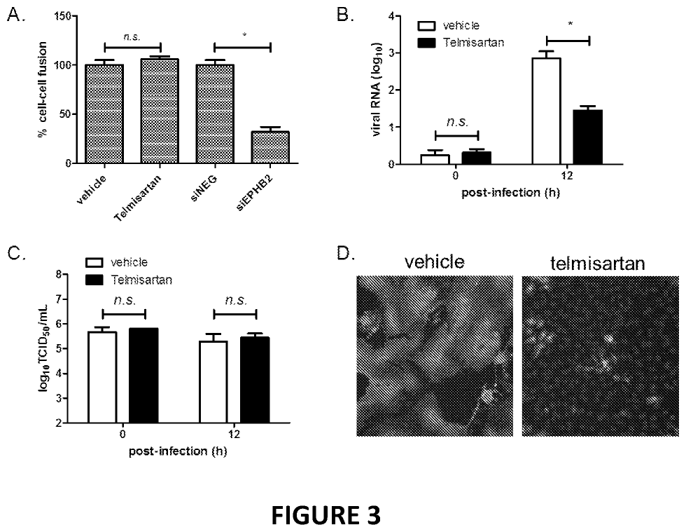

The stage of the henipavirus infection cycle that is blocked by Telmisartan was sought to be determined. The impact of Telmisartan on HeV cell entry was firstly determined using an established cell-cell fusion assay as described in Deffrasnes et al. (2016). When incubated with effector cells expressing HeV-F and HeV-G proteins, target cells treated with Telmisartan exhibited fusion 1 with effector cells comparable to target cells treated with vehicle (FIG. 3A), suggesting that Telmisartan does not block HeV cell entry. As a positive control, depleting cells of the HeV entry receptor ephrin-B2 (using a SMARTpool siRNA, siEFNB2) decreased cell-cell fusion by 70% relative to siNEG.

Whether Telmisartan blocks viral genomic replication and transcription was next investigated. The single-cycle replication kinetics of HeV in HeLa cells has been previously characterised as described in Deffrasnes et al. (2016). HeLa cells infected with a high MOI of HeV start producing infectious virions (above inoculum levels) between 12 and 24 h p.i. This indicates that the length of one cycle of HeV infection in HeLa cells is approximately 12 to 24 hours. Consequently, a validated TaqMan qPCR assay was used to measure intracellular viral RNA levels at 12 h p.i. In cells treated with vehicle, intracellular viral RNA levels increased ˜1000-fold above inoculum levels at 12 h p.i. (FIG. 3B). Within this single-cycle infection period (12 h p.i.), Telmisartan treatment significantly reduced intracellular viral RNA levels (FIG. 3B). Consistent with previous reports, virus titres were comparable between 12 h.p.i. and inoculum levels, confirming that the inhibition of viral RNA replication by Telmisartan occurred within the first cycle of virus infection (FIG. 3C). Furthermore, viral protein production (P protein and N protein) was almost completely abolished in HeV-infected cells treated with Telmisartan (FIG. 3D). Collectively, these results indicate that Telmisartan blocks viral RNA synthesis during the pioneering rounds of infection.

HeV Infection is Inhibited by Multiple Angiotensin II Receptor Antagonists:

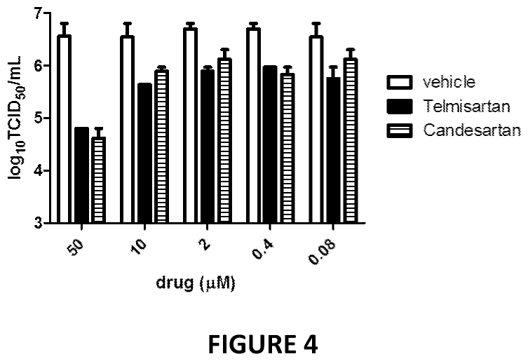

Whether the antiviral properties of Telmisartan are observed for other angiotensin II receptor antagonists was determined. It was found that Candesartan inhibited HeV infection in a dose-dependent manner, suggesting that angiotensin II receptor antagonists may inhibit virus growth more broadly (FIG. 4).

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All publications discussed and/or referenced herein are incorporated herein in their entirety.

The present application claims priority from Australian Provisional Patent Application No. 2017902236 filed on 13 Jun. 2017, which is hereby incorporated by reference in its entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Dalesandro et al. (1996) J. Thorac. Cardi. Surg. 11:416-422.
Deffrasnes et al. (2016) PLoS Pathog. 12: e1005478.
Foo et al. (2016) PLoS Pathog. 12:e1005974.
Good et al. (1966) Biochemistry 5:467.
Koc et al. (1996) Seminars in Oncology 23:46-65.
Langer (1990) Science 249: 1527-1533.
Makarov et al. (1996) Proc. Natl. Acad. Sci. USA 93:402-406.
Nolta et al. (1996) Proc Natl. Acad. Sci. USA 93:2414-2419.
Pager and Dutch (2005) J Virol, 2005. 79: 12714-12620.
Raper et al. (1996) Annals of Surgery 223:116-126.
Reed and Muench (1938) Am. J. Hygiene 27:493-497.

1. A method of treating or preventing a Mononegavirales viral infection in a subject, the method comprising administering an effective amount of an angiotensin II signalling inhibitor.

2. The method of claim 1, wherein the Mononegavirales viral infection is of a family selected from the group consisting of Pneumoviridae, Rhabdoviridae, Paramyxoviridae, and Filoviridae.

3. The method of claim 1 or claim 2, wherein the Mononegavirales viral infection is selected from the group consisting of Respiratory Syncytial Virus (RSV), Measles Virus (MeV), Hendra Virus (HeV), Nipah Virus (NiV), Avian Metapneumovirus (aMPV), Human Metapneumovirus (hMPV), Mumps Virus (MuV), Newcastle Disease Virus (NDV), Sendai Virus (SeV), Human Parainfluenza Virus 1 (HPIV-1), Maize Mosaic Virus (MMV), Rice Yellow Stunt Virus (RYSV), Lettuce Necrotic Yellow Virus (LNYV), Rabies Virus (RABV), Vesicular Stomatitis Indiana Virus (VSIV), Bovine Ephemeral Fever Virus (BEFV), Infectious Hematopoietic Necrosis Virus (IHNV), Marburg Virus (LVMV), and Ebola Virus (EBOV).

4. The method according to any one of claims 1 to 3, wherein the Mononegavirales viral infection is Respiratory Syncytial Virus (RSV).

5. The method according to any one of claims 1 to 3, wherein the Mononegavirales viral infection is Measles Virus (MeV).

6. The method according to any one of claims 1 to 5, wherein the angiotensin II signalling inhibitor has a structure selected from the group consisting of: 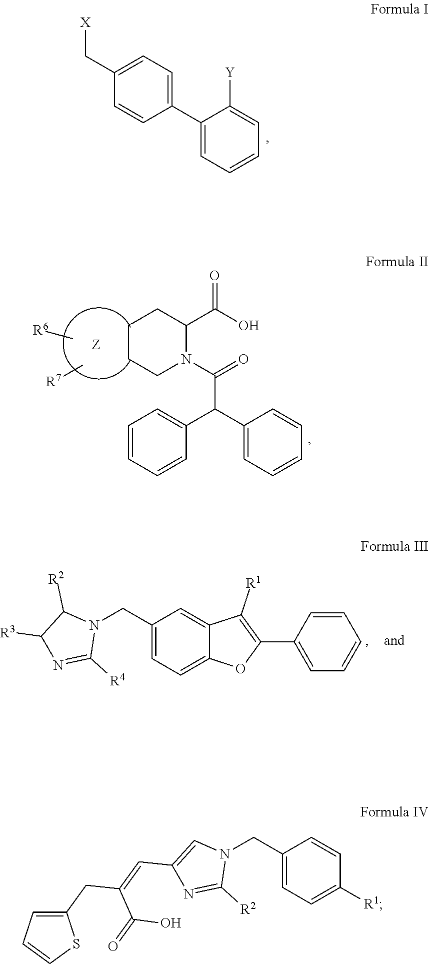
wherein X is selected from the group consisting of: 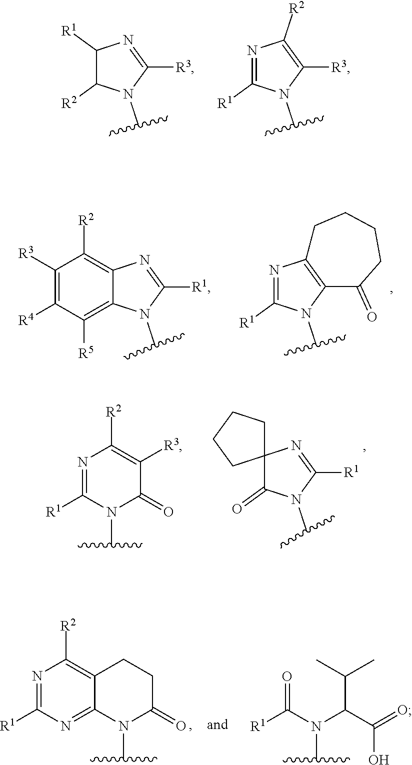
and wherein Y is selected from the group consisting of: 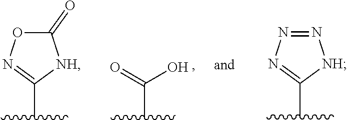
and wherein Z is a 5- or 6-membered monocyclic carbocyclic or monocyclic heterocyclic; and
wherein R¹, R², R³, R⁴, R⁵, R⁶ and R⁷ are each independently selected from the group consisting of hydrogen, halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, monocyclic or polycyclic carbocyclic, and monocyclic or polycyclic heterocyclic;
wherein the C₁₋₁₀alkyl, C₂₋₁₀alkenyl, monocyclic or polycyclic carbocyclic, and monocyclic or polycyclic heterocyclic, are each optionally substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, C₁₋₁₀alkyl, C₂₋₁₀alkenyl, monocyclic or polycyclic carbocyclic, and monocyclic or polycyclic heterocyclic;
wherein the monocyclic or polycyclic carbocyclic, and monocyclic or polycyclic heterocyclic are each optionally further substituted with one or more substituents selected from halogen, amino, hydroxyl, carboxyl, cyano, nitro, sulfonyl, aldehyde, alkanoyl, aroyl, alkanoate, aryloate, oxycarbonyl, aminocarbonyl, and C₁₋₁₀ alkyl; and
wherein the C₁₋₁₀alkyl and C₂₋₁₀alkenyl are each optionally interrupted with one or more heteroatoms independently selected from O, N and S.

7. The method according to any one of claims 1 to 6, wherein the angiotensin II signalling inhibitor is an angiotensin II receptor antagonist.

8. The method according to any one of claims 1 to 7, wherein the angiotensin II signalling inhibitor is an angiotensin II receptor AT₁ subtype antagonist.

9. The method according to any one of claims 1 to 8, wherein the angiotensin II signalling inhibitor is selected from the group consisting of Telmisartan, Candesartan, Losartan, Valsartan, Eprosartan, Irbesartan, Fimasartan, Saprisartan, Olmesartan, Azilsartan, Pratosartan, Tasosartan, EXP-3174, TCV-116, PD123319, EMA401, and pharmaceutically acceptable salts and solvates thereof.

10. The method according to any one of claims 1 to 9, wherein the angiotensin II signalling inhibitor is Telmisartan.

11. The method according to any one of claims 1 to 10, wherein the angiotensin II signalling inhibitor binds to the angiotensin II receptor.

12. The method according to any one of claims 1 to 5, wherein the angiotensin II signalling inhibitor is an angiotensin-converting enzyme (ACE) inhibitor.

13. The method according to any one of claims 1 to 5, wherein the angiotensin II signalling inhibitor is a renin inhibitor.

14. The method according to any one of claims 1 to 13, wherein the angiotensin II signalling inhibitor blocks viral RNA replication.

15. The method according to any one of claims 1 to 14, wherein the angiotensin II signalling inhibitor is administered in combination with another antiviral compound.

16. The method according to any one of claims 1 to 15, wherein the subject is not being treated for endothelial dysfunction, hypertension, diabetic nephropathy or congestive heart failure.

17. The method according to any one of claims 1 to 16, wherein the subject is not being treated for endothelial dysfunction.

18. The method according to any one of claims 1 to 17, wherein the angiotensin II signalling inhibitor is administered to the subject orally.

19. The method according to any one of claims 1 to 18, wherein the subject is a bird or mammal.

20. The method of claim 19, wherein the mammal is a human.

21. An angiotensin II signalling inhibitor when used in a method for treating or preventing a Mononegavirales viral infection in a subject.

22. Use of an angiotensin II signalling inhibitor in the manufacture of a medicament for the treatment or prevention of a Mononegavirales viral infection in a subject.
